SEQUENCE LISTING (6 sequences)

SEQ ID NO: 1 - DNA, 1171 bp, synthetic construct
  misc_feature 1..1171: note = Synthetic sequence
  source 1..1171: mol_type = other DNA
atgggcagcagccatcatcatcatcatcacagcagcggcctggtgccgcgcggcagccatatggctagcatgactggtggacagcaaatgggtcgcggatccgccattcctgcaccaactgacctgaagttcactcagggcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggacataagcttggcggcggaggcagtcacattccgcgagaagacgttgattatcatggcggtggcggttcacacatccctcgtgaggatgtcgactatcacgggggcggcggctcacatattccccgggaggatgtggattaccacggggggggcgggtctcatataccaagagaagatgtggactaccatggcggcggcggcagccatattccgcgcgaagatgtggattatcattaataactcgagcaccaccaccaccaccactgag

SEQ ID NO: 2 - protein, 387 aa, synthetic construct
  REGION 1..387: note = Synthetic sequence
MGSSHHHHHHSSGLVPRGSHMASMTGGQQMGRGSAIPAPTDLKFTQGTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGHKLGGGGSHIPREDVDYHGGGGSHIPREDVDYHGGGGSHIPREDVDYHGGGGSHIPREDVDYHGGGGSHIPREDVDYHLEHHHHHH

SEQ ID NO: 3 - protein, 15 aa, synthetic construct
  REGION 1..15: note = synthetic sequence
GGGGSHIPREDVDYH

SEQ ID NO: 4 - protein, 10 aa, synthetic construct
  REGION 1..10: note = synthetic sequence
HIPREDVDYH

SEQ ID NO: 5 - protein, 4 aa, synthetic construct
  REGION 1..4: note = synthetic sequence
GGGS

SEQ ID NO: 6 - protein, 5 aa, synthetic construct
  REGION 1..5: note = synthetic sequence
GGGGS

(19) United States
(12) Patent Application Publication
(10) Pub. No.: US 2026/0097149 A1
(43) Pub. Date: Apr. 9, 2026

(54) CELL FREE VASCULAR GRAFTS AND GRAFT MATERIALS FOR CELLULAR RECRUITMENT

(71) Applicant: The Research Foundation for the State University of New York, Amherst, NY (US)

(72) Inventors: Stelios ANDREADIS, East Amherst, NY (US); Randall SMITH, Lockport, NY (US); Bita NASIRI, Buffalo, NY (US)

(21) Appl. No.: 18/880,781

(22) PCT Filed: Jun. 26, 2023
(86) PCT No.: PCT/US2023/069071

§ 371 Date: Jan. 2, 2025

Related U.S. Application Data
(60) Provisional application No. 63/355,320, filed on Jun. 24, 2022.

(51) Int. Cl. A61L 27/34; A61L 27/54; C07K 14/00

(52) U.S. Cl. CPC A61L 27/34; A61L 27/54; C07K 14/001; A61L 2300/236; A61L 2300/25; A61L 2300/606; A61L 2430/20

(57) ABSTRACT

The present disclosure relates to an implantable vascular graft material, including: a substrate including a graft material, the substrate defining a top surface and a bottom surface; and one or more bispecific binding partners having a luminal binding domain bound to the top surface and one or more cellular binding domains. In embodiments, the disclosure includes an implantable vascular graft including: a tubular base layer including a graft material, the tubular base layer defining a luminal surface and an abluminal surface; and a fusion peptide having a heparin binding domain bound to the luminal surface and one or more monocyte binding domains. In embodiments, the present disclosure provides one or more implantable vascular grafts such as A-TEVs, methods of making vascular grafts, methods of use, and the like.

### STATEMENT REGARDING FEDERALLY SPONSORED RESEARCH OR DEVELOPMENT

This invention was made with government support under Agreement Nos. HL086582 and HL151196 awarded by The National Institutes of Health. The government has certain rights in the invention.

### SEQUENCE LISTING

The instant application contains a sequence listing which has been submitted electronically in ASCII format and is hereby incorporated by reference in its entirety. Said ASCII copy, created on 22 Jun. 2023, is named 030-7464WO01 and is 10,135 bytes in size.

### FIELD OF THE INVENTION

The present disclosure is generally related to the field of cell-free tissue engineered vascular grafts suitable for use in cellular recruitment such as monocyte recruitment to promote anti-occlusive and/or self-endothelialization capacity.

### BACKGROUND

Cardiovascular disease is the leading cause of death in the United States, claiming over 600,000 lives annually. Coronary artery disease is the most common form, with over 350,000 bypass grafting procedures performed every year, estimated at a total of $26 billion annually in healthcare costs, according to the American Heart Association. Tissue engineering approaches using native or synthetic scaffolds, or even scaffold-free strategies, have developed functional and implantable tissue engineered vessels (TEVs) that have been tested in small and large animal models. However, development of such TEVs typically requires the use of autologous cells, and weeks to months of cell expansion, tissue growth, and mechanical preconditioning before implantation. As a result, several laboratories have turned their attention to engineering cell-free vascular grafts. Due to lack of endothelial cells (EC), acellular (A)-TEV are more prone to acute thrombosis post implantation necessitating strategies that promote anti-occlusive and self-endothelialization capacity.

Several studies promoted endothelialization of the grafts by functionalizing the A-TEV lumen to attract rare circulating endothelial progenitor cells (EPCs) from blood or migration of adjacent EC from anastomotic sites to endothelialize the graft lumen. Others attempted to use the immune system to promote regeneration. Release of monocyte chemotactic protein-1 (MCP-1) from the graft caused rapid recruitment of immune cells to the scaffold promoting endothelialization. Coating a layer of IL4 on electrospun polycaprolactone (PCL) scaffolds promoted recruitment of anti-inflammatory M2 macrophages, resulting in significant reduction of foreign body encapsulation and neointimal hyperplasia post implantation.

To address these limitations, acellular (A)-TEVs have been developed using a native biodegradable material, small intestinal submucosa (SIS), which was sequentially coated with heparin and heparin-bound vascular endothelial growth factor (VEGF) to prevent thrombosis and promote endothelialization. These A-TEVs were tested in a preclinical ovine carotid model for up to 6 months demonstrating well over 90% patency. A functional confluent endothelium was formed in the lumen as early as one month post implantation. Most recently, VEGF based A-TEVs integrated seamlessly with the native vasculature and grew with the host, when implanted into neonatal lambs, suggesting that they might be suitable for treatment of congenital heart disorders to alleviate the need for repeated surgeries, currently the standard practice for pediatric patients.

Implanting VEGF based A-TEVs into the mouse abdominal aorta demonstrated the presence of anti-inflammatory, pro-regenerative M2 macrophages in the lumen and vascular wall, resulting in well-demarcated luminal and medial layers. On the other hand, control A-TEVs with heparin coated lumen were populated primarily by pro-inflammatory, M1 macrophages and the EC and SMC layers were not well defined, demonstrating the importance of the macrophage phenotype for proper vascular regeneration. Further examination of endothelialization by host cells showed that at one-week post-implantation into the sheep carotid artery, the graft lumen was populated with cells expressing monocyte (MC) markers, but one month later the luminal cells co-expressed MC and EC specific proteins, suggesting that MC might have differentiated into EC.

Prior art of interest includes U.S. Patent Publication No. 20210220523 (herein incorporated entirely by reference), however the grafts therein are different than the grafts of the present disclosure.

There remains a need for improved graft material and vascular grafts such as A-TEVs that overcome the aforementioned deficiencies.

### SUMMARY

Embodiments of the present disclosure provide implantable graft material such as vascular grafts, A-TEVs, methods of making vascular grafts, methods of use, and the like.

In embodiments, the present disclosure relates to an implantable vascular graft material, including: a substrate including a graft material, the substrate defining a top surface and a bottom surface; and one or more bispecific binding partners having a luminal binding domain bound to the top surface and one or more cellular binding domains. In embodiments, the one or more cellular binding domains are selected from monocyte binding domains, endothelial-like binding domains, and combinations thereof. In embodiments, the one or more subluminal binding domains are disposed upon the bottom surface.

In embodiments, the present disclosure relates to an implantable vascular graft including: a tubular base layer including a graft material, the tubular base layer defining a luminal surface and an abluminal surface; and a fusion peptide having an extracellular matrix, ECM, luminal surface and/or heparin binding domain bound to the luminal surface and one or more monocyte binding domains. In embodiments, the fusion peptide includes an ECM binding domain e.g., collagen binding domain and one or more monocyte binding domains. In embodiments, the fusion peptide is synthetic and includes a first predetermined binding domain and a second predetermined binding domain, wherein the second predetermined binding domain is different than the first predetermined binding domain.

In embodiments, the present disclosure relates to a method of making an implantable vascular graft including binding a tubular base layer including a graft material to a fusion peptide including one or more monocyte binding domains, wherein the tubular base layer defines a luminal surface and an abluminal surface, and wherein the fusion peptide includes an ECM or luminal surface binding domain, and/or heparin binding domain bound to the luminal surface.

In embodiments, the present disclosure relates to a method of administering a vascular graft to an endoluminal surface of a vessel of a subject in need thereof, the method including: intraluminally inserting a vascular graft of the present disclosure and positioning the vascular graft at a location in the vessel by use of a positioning apparatus; and anchoring the vascular graft at the location in the vessel of the subject.

In embodiments, the present disclosure relates to a method for recruiting monocytes in a vascular graft, including administering a vascular graft to a subject in need thereof such that a blood flow passes through the vascular graft; and providing a target for monocyte cell recruitment within the vascular graft, wherein the target is a fusion peptide and/or any molecule that binds monocytes, including one or more monocyte binding domains.

In embodiments, the present disclosure relates to an expression cassette and a fusion or multi-domain peptide generated from the expression cassette.

In embodiments, the present disclosure relates to DNA or cDNA that encodes a fusion or multi-domain peptide of the present disclosure.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting and non-exhaustive features will be described with reference to the following figures, wherein like reference numerals refer to like parts throughout the various figures. The figures below were not intended to be drawn to any precise scale with respect to size, angular relationship, or relative position.

FIGS. 1A-1D depict fusion peptide (H2R5) production, purification and activity. FIG. 1A depicts SDS Page. Lane A: ladder; B: bacterial lysate; C: flow-through of lysate from the heparin column; D: collection of washout through the column; E: H2R5 after release from the column and dialysis. The box encircles the band of purified H2R5 at MW of 40 kDa; FIG. 1B depicts ELISA for H2R5 immobilized on surface-bound heparin. Optical density (OD) of immobilized H2R5 as a function of input H2R5 surface concentration (nmole/cm²); FIG. 1C depicts a Western blot for pFAK and GAPDH for HUVECs. FIG. 1D depicts a graph showing quantification of western blot bands (n=4) normalized to GAPDH and the H2R5 band intensity at 30 min. (*) denotes statistical significance between H2R5 and heparin surface at each time point (n=4, P<0.05).

FIGS. 2A-2D depict endothelial cell capture and spreading on fusion peptide of the present disclosure such as H2R5. FIG. 2A depicts HUVEC capture efficiency (% cells bound) as a function of H2R5 concentration. Surface with immobilized FN or VEFG served as positive controls. (*, $) denote statistical significance of the indicated samples as compared to 0 mole/cm² (*) or 10 nmole/cm² ($) (n=3, p<0.05). FIG. 2B depicts representative actin phalloidin staining images of HUVECs about to the indicated surface concentration of H2R5, FN or VEGF (scale bar: 100 μm). Graphs show quantitative measurement of HUVECs area as a function of H2R5 surface concentration. Surface with immobilized FN or VEFG served as positive controls. (*, $) denotes statistical significance between the indicated sample and control surface, H2R5: 0 mole/cm² (*) or H2R5: 10 nmole/cm² ($) (n=3, p<0.05). FIG. 2C depicts quantification of HUVECs captured under flow on microfluidic channel functionalized with H2R5 or VEGF. Heparin alone served as negative control. (*) denotes significant difference between the indicated sample and heparin control for each shear stress (n=3, p<0.05). FIG. 2D depicts data relating to cell area and H2R5 surface concentration.

FIGS. 3A-3E depicts the capture and characterization of cells from PBMNCs on immobilized fusion peptide (e.g., H2R5). FIG. 3A depicts bar graphs shows PBMNC capture efficiency on immobilized fusion peptide (H2R5) as a function of H2R5 concentration. Surface with immobilized FN or VEFG served as positive controls. (*, $) denote statistical significance of the indicated samples as compared to 0 mole/cm² (*) or 10 nmole/cm² ($) (n=3, p<0.05). FIG. 3B depicts the capture efficiency of PBMNC pre-treated with α4β1 blocking antibody at the indicated concentrations (μg/ml) before placed on H2R5 functionalized surface. (*) denotes statistical significance between the indicated samples (n=3, p<0.05). FIG. 3C depicts the multi-color flow cytometry assessment of PBMNCs isolated directly from whole blood; or after 1 hr capture on immobilized H2R5 surface; or at 24 h after capture on immobilized H2R5. FSC/SSC shows the entire population present in PBMNCs or adhered on fusion peptide H2R5 for 1 hr or 24 hr. (blue and red dots). Blue population denotes CD14⁺ cells. Green population denotes classical MC (CD16⁻); and black population indicates non-classical MC (CD16⁺). FIG. 3D depicts bar graph indicating the number of the PBMNCs captured under flow on microfluidic channel functionalized with fusion peptide H2R5, VEGF or heparin alone. (*) denotes significant difference between the indicated H2R5/VEGF surfaces and the heparin surface. (#) denotes significant difference between fusion peptide H2R5 and VEGF surfaces 10 dynes/cm² shear stress (n=3, p<0.05). FIG. 3E depicts the representative image of PBMNCs captured on H2R5 functionalized microfluidic channel and immunostained for the MC marker, CD14.

FIG. 4 depicts gene expression of PBMNCs captured on fusion peptide (H2R5) surface. Real time RT-PCR forth indicated MC, M1 or M2 genes of MC plated on H2R5 surface for 1 hr. or 24 hr. RPL32 served as housekeeping gene and all data were normalized to the expression level of PBMNCs before plating (t=0 hr). (*) indicates significant difference as compared to the same donor after 1 hr of adhesion (n=3, p<0.05).

FIGS. 5A-5E depict A-TEV implantation and histological assessment. FIG. 5A depicts images of H2R5 A-TEVs prior to implantation, at implantation, and explanted grafts at 4- and 12-weeks post-implantation. Black arrows represent ends of aorta and anastomotic sites. Scale bars: 2 mm. FIG. 5B depicts color doppler ultrasound images of A-TEV at the indicated times post-implantation. FIG. 5C depicts gross representative images of explanted A-TEVs at 4 and 12 weeks show clearly patent lumens. FIG. 5D depicts representative H&E images of cross sections from the middle of A-TEV explants at 4 and 12 weeks post-implantation. Bottom images represent higher magnification of the areas delineated by squares. Top images scale bar: 200 μm; Bottom images scale bar: 50 μm; L: lumen. FIG. 5E depicts enface images of the lumen of A-TEV explants at 4 weeks post-implantation. Red arrow indicates the direction of blood flow (scale bar: 50 μm).

FIGS. 6A and 6B depict host cells populating the lumen of A-TEVs express endothelial and smooth muscle markers. A-TEVs were explanted at 4 weeks post-implantation. FIG. 6A depicts immunostaining for eNOS and α-SMA. Scale bar: 50 μm.

FIG. 6B depicts immunostaining for Ki67 and eNOS or Ki67 and α-SMA. Scale bar: 50 μm; L: lumen.

FIGS. 7A and 7B depict host cells populating the lumen of A-TEVs co-express MC/MΦ and EC or SMC markers. A-TEVs were explanted at 4 weeks post-implantation. FIG. 7A depicts immunostaining for the MC marker CD14 (green) and eNOS or α-SMA (red). FIG. 7B depicts immunostaining for the M2 marker CD206 (green) and eNOS or α-SMA (red). Scale bar: 50 μm. L: lumen.

FIGS. 8A-8C depict lineage tracing of host cells recruited to the A-TEV lumen. A-TEV implanted in CX3CR1-Confetti mice were explanted at 2- and 4-weeks post implantation. FIG. 8A depicts representative images of tissue sections from the middle of the grafts showing fluorescently labeled cells adhered on the graft lumen. Immunostaining of tissue sections for FIG. 8B depicts CD14; or FIG. 8C depicts CX3CR1. Scale bar: 50 μm; L: lumen.

FIGS. S1A and S1B present data indicating that H2R5 is more efficient in preventing platelet adhesion as compared to H2RGD5. Non-tissue culture plates (A) or SIS (B) were functionalized with H2R5, H2RGD5 or heparin alone and treated with platelet rich plasma for 60 min. FIG. S1A presents immunostaining for activated platelet marker CD62 (red). Scale bar: 100 μm. FIG. S1B presents SEM images SIS sheet. Scale bar: 20 μm.

FIG. S2 presents a schematic describing preparation of H2R5 decorated A-TEV.

FIGS. S3A and S3B present host cells populating the lumen of A-TEVs express endothelial and smooth muscle markers. A-TEVs were explanted at 12 weeks post-implantation. FIG. S3A presents immunostaining for eNOS and α-SMA. Scale bar: 20 μm. FIG. S3B presents immunostaining for Ki67 and eNOS; or (C) Ki67 and α-SMA. Scale bar: 20 μm. L: lumen.

FIG. S4 presents cells recruited to the media layer co-express α-SMA and MYH-11. A-TEVs were explanted at 4- or 12-weeks post-implantation. Immunostaining for MYH11 (red) and α-SMA (green). Scale bar: 20 μm. L: lumen.

FIGS. S5A and S5B present host cells populating the lumen of A-TEVs co-express MC/Mφ and EC or SMC markers even after 3 months in vivo. A-TEVs were explanted at 12 weeks post-implantation. FIG. S5A presents immunostaining for the MC marker CD14 (red) and eNOS or α-SMA (green). FIG. S5B presents immunostaining for the M2 marker CD206 and eNOS or α-SMA. Scale bar: 20 μm. L: lumen.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

In embodiments, the present disclosure includes an implantable vascular graft such as an A-TEV. In embodiments, the implantable vascular graft includes: a tubular base layer including a graft material, the tubular base layer defining a luminal surface and an abluminal surface, and a fusion or multi-domain peptide having a heparin binding domain bound to the luminal surface and one or more monocyte binding domains. Advantages of the vascular grafts such as A-TEVs of the present disclosure include providing one or more cell-free tissue engineered vascular grafts suitable for use in monocyte recruitment to promote anti-occlusive and self-endothelialization capacity.

In embodiments, a method to recruit monocytes (MC) from blood to regenerate vascular tissue from unseeded (cell-free) tissue engineered vascular grafts such as A-TEVs is provided. In embodiments, a fusion peptide, is immobilized on the surface of one or more vascular grafts, to capture one or more blood derived MCs under static or flow conditions in a shear stress dependent manner. In embodiments, bound MC turns into macrophages (Mc) expressing both M1 and M2 phenotype specific genes. In embodiments, a functionalized A-TEV includes a fusion peptide of the present disclosure implanted into a vessel, such as a subject's aorta, and remains patent while forming a continuous endothelium expressing both EC and MC specific proteins. In embodiments, underneath an EC layer, multiple cells layers are formed co-expressing both smooth muscle cell (SMC) and MC specific markers.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

The term “about”, as used herein, refers to +/−10% of the stated value or a chemical or obvious equivalent thereof.

“Binding” as used herein (e.g., with reference to a heparin-binding domain or a monocyte binding domain of a polypeptide, binding to a target nucleic acid, and the like) refers to a non-covalent interaction between macromolecules (e.g., between a protein and a target). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻¹¹ M, less than 10⁻¹² M, or less than 10⁻¹⁵ M. “Affinity” refers to the strength of binding, increased binding affinity being correlated with a lower K_{d}.

By “binding domain” it is meant a protein domain that is able to bind, for example covalently or non-covalently, to another molecule. A binding domain can bind to, for example, a target molecule and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety.

In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences.

The term “isolated” means a substance in a form or environment that does not occur in nature. In embodiments, one or more peptides may be synthetically produced and are considered isolated for purposes of the present disclosure, as are native or one or more fusion peptides of the present disclosure, which have been separated, fractionated, or partially or substantially purified by any suitable technique.

As used herein, “endoluminally,” “intraluminally” or “transluminal” all refer synonymously to implantation placement by procedures wherein the prosthesis is advanced within and through the lumen of a body vessel from a remote location to a target site within the body vessel. In vascular procedures, a medical device will typically be introduced “endovascularly” using a catheter over a wire guide under fluoroscopic guidance. The catheters and wire guides may be introduced through conventional access sites to the vascular system. It is also contemplated that embodiments disclosed herein include interpositional implantation or end-to-end, or end-to-side or side-to-side implantation of devices that are inclusive of grafts.

As used herein, the term “forming a mixture” refers to the process of bringing into contact at least two distinct species such that they mix together and interact. In embodiments, a “mixture” refers to a combination of two or more substances in which each substance retains its own chemical identity and properties. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

The terms “frame” and “support frame” are used interchangeably herein to refer to a structure that can be implanted, or adapted for implantation, within the lumen of a body vessel.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

Unless otherwise indicated, as used herein, a “layer” refers to a portion of a structure having a defined composition or structure and a defined boundary with respect to an adjacent material.

The term “luminal surface” or “luminal side,” as used herein, refers to the portion of the surface area of a medical device or vascular graft defining at least a portion of an interior lumen. Conversely, the term “abluminal surface” or “abluminal side,” as used herein, refers to portions of the surface area of a medical device or vascular graft that do not define at least a portion of an interior lumen. For example, where the medical device or vascular graft is a tubular frame defining a cylindrical lumen, the abluminal surface can include the exterior surface, sides and edges of the tubular frame, while the luminal surface can include the interior surface of the tubular frame.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

As used herein, the terms “vessel” or “body vessel” mean any body passage lumen that conducts fluid, including but not limited to blood vessels, heart vessels, esophageal, intestinal, biliary, urethral and ureteral passages. The vessels can include a vein, an artery, a biliary duct, a ureteral vessel, a portion of the alimentary canal, and other bodily vessels.

These definitions are intended to supplement and illustrate, not preclude, the definitions that would be apparent to one of ordinary skill in the art upon review of the present disclosure. Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Detailed Description of Certain Preferred Embodiments

In embodiments, the present disclosure includes an implantable vascular graft including: a tubular base layer including a graft material, the tubular base layer defining a luminal surface and an abluminal surface; and a fusion or multi-domain peptide having a heparin binding domain bound to the luminal surface and one or more monocyte binding domains. In embodiments, the vascular graft includes acellular tissue engineered vascular grafts (such as A-TEVs) that do not require cell seeding, advantageously avoiding many problems associated with seeding, including the need for an invasive procedure to obtain autologous cells, the need for a substantial period of time in order to expand the cells in culture, the inherent difficulty in obtaining healthy autologous cells from diseased donors, and an increased risk of contamination and the potential for dedifferentiation of the cells. In embodiments, cell-free A-TEVs or grafts thereof therefore have excellent off-the-shelf availability and excellent overall clinical utility.

In embodiments, the present disclosure includes an implantable vascular graft material, including: a substrate including a graft material, the substrate defining a top surface and a bottom surface; and one or more bispecific binding partners having a luminal binding domain bound to the top surface and one or more cellular binding domains. In embodiments, the one or more cellular binding domains are selected from monocyte binding domains, endothelial-like binding domains, and combinations thereof. In embodiments, one or more subluminal binding domains are disposed upon the bottom surface. In embodiments, the one or more cellular binding domains are monocyte binding domains including integrin α4β1 and/or VEGFR1. In embodiments, the one or more cellular binding domains are endothelial-like binding domains including Flk1 (VEGFR2). In embodiments, the luminal binding domain is characterized as an ECM binding domain, a heparin binding domain, a fibrin binding domain or a collagen binding domain. In embodiments, the bispecific binding partner is a polymer, or synthetic polymer. In embodiments, the bispecific binding partner is a biopolymer or synthetic biopolymer. In embodiments, the bispecific binding partner is an aptamer or synthetic aptamer, or combination of synthetic aptamers. In embodiments, the bispecific binding partner is a peptide having at least 80 percent sequence identity to SEQ ID. No. 2. In embodiments, the bispecific binding partner is a peptide having at least 90%, at least 95%, at least 97%, or at least 99 percent sequence identity to SEQ ID. No. 2. In embodiments, the one or more one or more cellular binding domains are monocyte binding domains configured to recruit monocytes when contacted with blood. In embodiments, the implantable vascular graft material is suitable for use in a blood contacting tissue including heart leaflet, heart patch, or tubular vascular graft, and the like.

In some embodiments, the present disclosure includes an implantable vascular graft including: a tubular base layer including a graft material, the tubular base layer defining a luminal surface and an abluminal surface, and one or more bispecific binding partners having a luminal binding domain bound to the luminal surface and one or more cellular or monocyte binding domains. In embodiments, the bispecific binding partner is one or more of a polymer, a biopolymer, an aptamer, or combinations thereof. In embodiments, the bispecific binding partner is a fusion or multi-domain peptide having a luminal binding domain characterized as a heparin binding domain. In embodiments, a chitosan-heparin layer is disposed directly atop the luminal surface and between the luminal surface and the fusion or multi-domain peptide. In embodiments, the one or more bispecific binding partners is a fusion protein is characterized by the formula H2R5, wherein H2 is a heparin binding domain of fibronectin, and R5 includes a plurality of tandem repeats, wherein each tandem repeat comprises a flexible linker motif followed by a peptide including the sequence HIPREDVDYH. In embodiments, R5 is -(GGGS-HIPREDVYH)₅. In embodiments, the flexible linker motif is the polypeptide sequence GGGS. In embodiments, the peptide including the sequence HIPREDVDYH binds to integrin α4β1 on a cell surface. In embodiments, the graft material is a biodegradable material characterized as small intestinal submucosa (SIS). In embodiments, the graft material is an elastomer. In embodiments, the fusion peptide is characterized as multivalent and binds one or more circulating monocytes when disposed within a blood stream. In embodiments, the vascular graft, upon implantation, promotes endothelialization of the tubular base layer by recruiting one or more monocytes or one or more endothelial like cells. In embodiments, the one or more monocyte binding domains are disposed opposite of the luminal domain and in contact with a blood flow, when present, through the tubular base layer. In embodiments, the monocyte binding domain binds integrin α4β1, or VEGFR1 expressed on the surface of a monocyte. In embodiments, the luminal binding domain is a heparin binding domain that binds heparin. In embodiments, the fusion peptide is configured to capture one or more blood derived monocytes under static or flow conditions in a shear stress dependent manner. In embodiments, the fusion peptide in combinations with one or more monocytes promotes the formation of a functional confluent endothelium in a lumen of the vascular graft. In embodiments, the graft material or grafts of the present disclosure upon implantation, a plurality of luminal cells co-express monocyte and endothelial cell proteins. In embodiments, the vascular graft has a length of about 1 mm to about 1000 mm, or about 1 mm to about 500 mm, or about 1 mm to about 150 mm. In embodiments, the vascular graft has a fully expanded inner diameter of about 0.5 mm to about 25 mm. In embodiments, the graft material or vascular grafts of the present disclosure include a therapeutic agent. In embodiments, the graft material or vascular grafts of the present disclosure include one or more anchoring means for anchoring the vascular graft to a surrounding blood vessel wall when the vascular graft is in an expanded state.

In embodiments, the present disclosure includes a method of making an implantable vascular graft including: binding a tubular base layer including a graft material and a fusion or multi-domain protein including one or more cellular or monocyte binding domains, wherein the tubular base layer defines a luminal surface and an abluminal surface, wherein the fusion or multi-domain peptide comprises a luminal binding domain bound to the luminal surface.

In embodiments, the present disclosure includes a method of administering a vascular graft or graft material to an endoluminal surface of a vessel of a subject in need thereof, the method including: intraluminal inserting a vascular graft or graft material according to the present disclosure and positioning the vascular graft at a location in the vessel by use of a positioning apparatus, and anchoring the vascular graft at the location in the vessel of the subject. In embodiments, the subject is a human. In embodiments, the vessel is selected from the group consisting of a vein, an artery, a biliary duct, a ureteral vessel, a body passage, a portion of an alimentary canal, a portion of the heart, or a portion of the aorta, and the like. In embodiments, the vascular graft or graft material has an increased patency as compared to a reference patency for the otherwise same vascular graft except where the vascular graft does not contain the fusion peptide, wherein the patency is measured at about the same period of time following administration in the otherwise same location of the otherwise same subject.

In embodiments, the present disclosure includes a method for recruiting predetermined cells such as monocytes in a vascular graft, including: administering a vascular graft or graft material to a subject in need thereof such that a blood flow passes through the vascular graft or graft material or contacts the vascular graft or graft material; and providing a target for cell or monocyte cell recruitment within the vascular graft or adjacent the graft material, wherein the target is a bispecific binding partner including one or more cellular binding domains such as a monocyte binding domains.

In embodiments, the present disclosure includes a synthetic peptide including an amino acid sequence having at least 90 percent sequence identity to SEQ ID NO: 2. In embodiments, the amino acid sequence has at least 95%, at least 97%, or at least 99% sequence identity to SEQ ID NO: 2. In embodiments, the synthetic peptide is characterized as isolated, purified, and/or substantially purified.

In embodiments, the graft material or vascular grafts of the present disclosure, upon implantation into a subject in need thereof, promotes differentiation into any tissue that can be regenerated such as tissues including a pro-regenerative macrophage phenotype, pro-regenerative immune cells, and the like.

In embodiments, an implantable vascular graft includes: a tubular base layer including a graft material. In embodiments, the tubular base layer is generally in a tube-like or tubular shape having a hollow interior, or flow through design. In embodiments, the tubular base layer has a length of about 1 mm to about 1000 mm, or about 2 mm to about 800 mm. In embodiments, the tubular base has a fully expanded inner diameter of about 0.5 mm to about 25 mm. In embodiments, the tubular base layer is a substrate upon which the vascular graft of the present disclosure is formed. Accordingly, the vascular graft of the present disclosure may also have identical or similar dimensions as the tubular base layer, such as a length of about 1 mm to about 1000 mm, or about 2 mm to about 800 mm, e.g., a fully expanded inner diameter of about 0.5 mm to about 25 mm. In embodiments, the tubular base layer defines a luminal surface and an abluminal surface.

In embodiments, the graft material may be any suitable material for making a graft. Non-limiting examples of graft material suitable for use herein includes a biodegradable graft material such as e.g., small intestinal submucosa (SIS). Additional non-limiting examples of a graft material included elastomers, synthetic elastomers, polymers, biopolymers, synthetic polymers, synthetic biopolymers, and the like.

In embodiments, a first binding agent may be deposited directly atop the luminal surface. For example, where the graft material is SIS, chitosan may be deposited or coated atop the SIS graft material to form a chitosan layer thereon. In embodiments, the chitosan layer may entirely, or partially cover or coat the luminal surface of the tubular base layer. In embodiments, the chitosan layer may be applied in an amount sufficient to form a chitosan layer having a thickness of 0.1 to 1 mm.

In embodiments, a second binding agent may be deposited directly atop the chitosan layer. For example, where the tubular base layer has a graft material of SIS, and chitosan is deposited atop the SIS graft material to form a chitosan layer thereon, a heparin layer may be deposited directly atop the chitosan to form a chitosan-heparin layer. In embodiments, the chitosan-heparin layer may entirely, or partially cover or coat the chitosan layer disposed atop the luminal surface of the tubular base layer. In embodiments, the chitosan-heparin layer may be applied in an amount sufficient to form a chitosan-heparin layer having a thickness of 0.1 to 1 mm.

In embodiments, a chitosan-heparin layer is deposited atop and/or affixed to the inner surface of the tubular base layer such that a peptide having a heparin binding domain binds to the luminal surface. In embodiments, the peptide is a fusion or multi-domain peptide and further includes one or more monocyte binding domains. In embodiments, a chitosan-heparin layer is disposed directly atop the luminal surface and between the luminal surface and the fusion or multi-domain peptide.

In embodiments, the fusion or multi-domain protein is characterized by the formula H2R5, wherein H2 is a heparin binding domain of fibronectin, and R5 (SEQ ID NO: 3) includes a plurality of tandem repeats (such as 1-5 tandem repeats), wherein each tandem repeat includes a flexible linker motif followed by a peptide comprising the sequence HIPREDVDYH (SEQ ID NO: 4). In embodiments, R5 is -(GGGS-HIPREDVYH)₅. In embodiments, the flexible linker motif is the polypeptide sequence GGGS or GGGGS. In some embodiments, the peptide including the sequence HIPREDVDYH (SEQ ID NO: 3) binds to integrin α4β1 on a cell surface. In some embodiments, the peptide is a fusion peptide characterized as bivalent or multivalent which binds one or more circulating monocytes when disposed within a blood stream.

In embodiments, the peptide is a fusion peptide having the amino acid sequence of: MGSSHHHHHHSSGLVPRGSHMASMTGGQQMGRGSAIPAPTDLKFTQGTPTSLSA QWTPPNVQLTGYRVRVTPKEKTGPMKEINLAPDSSSVVVSGLMVATKYEVSVYAL KDTLTSRPAQGVVTTLENVSPPRRARVTDATETTITISWRTKTETITGFQVDAVPAN GQTPIQRTIKPDVRSYTITGLQPGTDYKIYLYTLNDNARSSPVVIDASTAIDAPSNLRF LATTPNSLLVSWQPPRARITGYIIKYEKPGSPPREVVPRPRPGVTEATITGLEPGTEY TIYVIALKNNQKSEPLIGHKLGGGGSHIPREDVDYHGGGGSHIPREDVDYHGGGGS HIPREDVDYHGGGGSHIPREDVDYHGGGGSHIPREDVDYH**LEHHHHHH* (SEQ ID NO: 2) Non-limiting examples of suitable fusion or multi-domain peptide include peptides having at least 80%, at least 90%, at least 95%, at least 97%, or at least 99% identity to SEQ ID NO.2. In embodiments, the fusion peptide can have, 1, 2, 3, 4 or more conservatively substituted amino acids. In embodiments, the fusion peptide may have several (such as 2-4), or a plurality (such as 2-5, or 2 or more) conservative substitutions. In embodiments, functional fragments of peptides of the present disclosure include the peptides mentioned herein including those fragments that bind heparin and one or more monocytes of interest. In embodiments, function fragments include a first binding domain suitable for binding to the luminal wall such as a heparin binding domain, and a second binding domain such as a monocyte binding domain. In embodiments, the peptide or functional fragment thereof includes a several (such as 2-4), or a plurality (such as 2-5, or 2 or more) monocyte binding domains. In embodiments, the peptide of the present disclosure such as a fusion peptide, di, multivalent peptide may have one or more histidine tags located at the C-terminus of the peptide or amino acid sequence. For example, 2-6 histidine amino acid residues may be at the C-terminus of the peptide, fusion peptide, or each R5 as described herein.

In embodiments, the fusion protein of the present disclosure is encoded by a DNA or cDNA. A non-limiting example of DNA encoding a peptide of the present disclosure is shown in SEQ ID NO:1.

In embodiments, the vascular graft, upon implantation, promotes endothelialization of the tubular base layer by recruiting one or more monocytes. In embodiments, the one or more monocyte binding domains are disposed (positioned?) opposite of the heparin binding domain and in contact with a blood flow, when present, through the tubular base layer. In embodiments, the monocyte binding domain binds integrin α4β1 expressed on the surface of a monocyte. In embodiments, the heparin binding domain binds heparin. In embodiments, the fusion or multi-domain peptide is configured to capture one or more blood derived monocytes under static or flow conditions in a shear stress dependent manner.

In embodiments, fusion or multi-domain peptide in combinations with one or more monocytes promotes the formation of a functional confluent endothelium in a lumen of the vascular graft. In embodiments, a vascular graft of the present disclosure, upon implantation, provides anti-inflammatory, pro-regenerative M2 macrophages disposed in a vascular graft wall, resulting in one or more luminal and medial layers. In embodiments, upon implantation, a plurality of luminal cells co-express monocyte and endothelial cell proteins.

In embodiments, an implantable vascular graft of the present disclosure further includes a therapeutic agent.

In embodiments, an implantable vascular graft of the present disclosure, further includes one or more anchoring means for anchoring the vascular graft to a surrounding blood vessel wall when the vascular graft is in an expanded state. For example, a suture or surgical staple may be suitable anchors of the present disclosure for holding the vascular graft in a predetermined or desired location in a subject depending upon surgical needs.

Methods of Use: In embodiments, the vascular grafts and prostheses described herein can be delivered to any suitable body vessel, including a vein, artery, biliary duct, ureteral vessel, body passage or portion of the alimentary canal. Methods for delivering vascular grafts and prostheses as described herein to any suitable body vessel are also provided, such as a vein, artery, biliary duct, ureteral vessel, body passage or portion of the alimentary canal. While many aspects discussed herein described the implantation of vascular grafts and prostheses in a vein or artery, other aspects provide for implantation within other body vessels. In another matter of terminology there are many types of body canals, blood vessels, ducts, tubes and other body passages, and the term “vessel” is meant to include all such passages.

In embodiments, the methods of the present disclosure include deploying the vascular grafts and prostheses in a vessel involves radially compressing and loading the vascular grafts and prostheses into a delivery device, such as a catheter. A restraining means may maintain the vascular grafts and prostheses in the radially compressed configuration. For example, a self-expanding stent graft may be retained within a slidable sheath, while stent grafts that are not self-expanding may be crimped over a balloon portion of a delivery catheter. The compressed stent graft is thereby mounted on the distal tip of the delivery device, translated through a body vessel on the delivery device, and deployed from the distal end of the delivery device. For example, a delivery device may be a catheter having a pushing member adapted to urge the stent graft away from the delivery catheter. A sheath may be longitudinally translated relative to the stent graft to permit the stent graft to radially self-expand at the point of treatment within a body vessel. Alternatively, a balloon may be inflated to radially expand the stent graft. Additionally, the methods of the present disclosure include typical procedures of arteriovenous grafts, e.g., for kidney dialysis patients and typical CABG (Carotid Artery Bypass Grafts) for patients with cardiac diseases.

Methods of treating a subject, which can be animal or human, are also provided. The methods can include the step of implanting one or more vascular grafts or prostheses as described herein. Methods of treatment can include the step of implanting one or more vascular grafts or prostheses configured to release a therapeutic agent, as described herein. In some embodiments, methods of treating may also include the step of delivering a vascular graft or prosthesis to a point of treatment in a body vessel, or deploying a vascular graft or prosthesis at the point of treatment.

Methods can include administering a vascular graft to an endoluminal surface of a vessel of a subject in need thereof, by providing a vascular graft described herein, intraluminally inserting the vascular graft and positioning the vascular graft at a location in the vessel expanding and anchoring the vascular graft at the location in the vessel of the subject. The vessel can include a vein, an artery, a biliary duct, a ureteral vessel, a body passage, or a portion of the alimentary canal.

In embodiments, the methods result in a vascular graft having an increased patency as compared to a reference patency for the otherwise same vascular graft except where the vascular graft does not contain the or multi-domain fusion peptide, wherein the patency is measured at about the same period of time following administration in the otherwise same location of the otherwise same subject.

In embodiments, the disclosed compositions and methods are based on the finding that altering the recruitment of monocytes and the infiltration of macrophages in the scaffold or sidewall of the A-TEV is beneficial. Methods for increasing the patency of biodegradable, synthetic vascular grafts are therefore disclosed. A-TEV in combination with a fusion peptide configured to bind to the A-TEV and one or more monocytes of interests is also disclosed.

In embodiments, the A-TEVs of the present disclosure may be used as venous, arterial or artero-venous conduits for any vascular or cardiovascular surgical application. Exemplary applications include, but are not limited to, congenital heart surgery, coronary artery bypass surgery, peripheral vascular surgery and angioaccess.

In embodiments, the present disclosure relates to a method of making an implantable vascular graft of the present disclosure, the method including: binding a tubular base layer including a graft material and a fusion or multi-domain protein including one or more monocyte binding domains, wherein the tubular base layer defines a luminal surface and an abluminal surface, wherein the fusion or multi-domain peptide comprises a heparin binding domain bound to the luminal surface.

In embodiments, the present disclosure includes a method of administering a vascular graft to an endoluminal surface of a vessel of a subject in need thereof, the method including: intraluminally inserting a vascular graft of the present disclosure and positioning the vascular graft at a location in the vessel by use of a positioning apparatus; and anchoring the vascular graft at the location in the vessel of the subject. In embodiments, the subject is a human. In embodiments, the vessel is selected from the group consisting of a vein, an artery, a biliary duct, a ureteral vessel, a body passage, a portion of an alimentary canal, a portion of the heart, or a portion of the aorta. In embodiments, the vascular graft has an increased patency as compared to a reference patency for the otherwise same vascular graft except where the vascular graft does not contain the fusion peptide, wherein the patency is measured at about the same period of time following administration in the otherwise same location of the otherwise same subject.

In embodiments, the present disclosure includes a method for recruiting monocytes in a vascular graft, including: administering a vascular graft to a subject in need thereof such that a blood flow passes through the vascular graft; and providing a target for monocyte cell recruitment within the vascular graft, wherein the target is a fusion peptide comprising one or more monocyte binding domains.

### EXAMPLES

### Example 1

Recruitment of blood MCs to the surface of A-TEVs is presented by mimicking the mechanism through which circulating MC interact with the endothelium. Direct evidence of MC recruitment on the graft lumen using a novel mouse model that enables tracing of blood MC populating the graft is also provided.

As described further below, lineage tracing analysis using a novel CX3CR1-confetti mouse model demonstrated that fluorescently labeled MC populated the graft lumen by two and four weeks post-implantation, demonstrating MC/MΦ recruitment to the graft lumen. Given the abundance of circulating MCs in blood, blood is an excellent source of cells that contribute directly to the endothelialization and vascular wall formation of acellular vascular grafts such as A-TEVs under the right chemical conditions and biomechanical cues.

### Materials and Methods

### H2R5 Cloning and Protein Production

The H2R5 sequence H2-(GGGS-HIPREDVYH)₅ consists of two parts: (i) H2, the second heparin binding domain of fibronectin; (ii) R2, five tandem repeats, each including a flexible linker motif, GGGS followed by a peptide HIPREDVDYH from CS-5 region of fibronectin, which is known for binding to integrin α4β1. The H2 domain was cloned by RT-PCR of the second heparin binding domain of fibronectin using cloning primers (Table 1) containing the BamHI and XhoI cutting sites and inserted in pET28a expressing vector.

TABLE
Cloning primer

| | |
|---|---|
| Forward_H2_ | ATATGGATCCgccATTCCTGCACCAACT |
| BamHI | GACCTGAAGTTCA |
| Reverse_H2_ | ATATAAGCTTATGTCCAATCAgGGGCTC |
| HindIII | GCTCTTCT |

For R5, a pair of complementary oligonucleotides—one containing Hi dIII and the other the XhoI cutting site—were purchased from Invitrogen (Waltham, MA), annealed and cloned to the same sites after the H2 sequence in the pET28a vector. The H2RGD5, H2-(GGGS-GRGDS)5 fusion protein was produced in a similar manner as described from the group.[34] The H2R5 protein was produced in bacterial strain E. coli BL21-DE3pLysis. Specifically, bacteria were expanded until the OD reached to OD=0.7 and then induced with 0.1 mm IPTG (Sigma-Aldrich. St. Louis, MO) for protein production overnight at 22° C. and 240 rpm. Next day, the bacteria were centrifuged at 4000 rpm for 20 min and pellets resuspended in lysis buffer (500 mm NaCl, VWR Chemicals, LLC, Solon, OH, USA), in 1×PBS, pH 7.4), containing with 1 mg mL-1 lysozyme (Sigma-Aldrich, St. Luis, MO), 1% Triton X-100 (Sigma-Aldrich. Inc. St. Luis, MO), and 1 mm PMSF (Sigma-Aldrich. Inc. St. Luis, MO) as protease inhibitor stirred for 1 h at room temperature (RT) following by sonication for 10 cycles with 50% intensity, 30 s on/30 s off. The soluble protein was obtained by ultracentrifugation of sonicated lysate at 50 000×g for 15 min using the Avanti high performance centrifuge (Beckman Coulter Inc. Indianapolis, IN). H2R5 protein was then purified using HisTrapTMHP Column (Cytvia, Uppsala, Sweden) according to the manufacturer's instructions. The final concentration was measured by Bradford and the protein purity was tested using 10% DS-PAGE where H2R5 was apparent at molecular weight of ≈42 kDa.

### Immobilization of H2R5

Non-tissue culture plates were coated with 0.1 mg/ml chitosan oligosaccharide lactate, average Mn 5,000 (Sigma-Aldrich. Inc. St. Luis, MO) in sterile DI water, overnight at RT. Surfaces were washed twice with DI water to remove excess chitosan and then treated with 1 mg/ml heparin sodium salt from porcine intestinal mucosa (Sigma-Aldrich. Inc. St. Luis, MO) and dissolved in sterile DI water for 2 hr at 37° C. Finally, H2R5 diluted in PBS was added on chitosan-heparin surfaces at 37° C. for 2 hr after washing the excess heparin with 1×PBS. Binding of H2R5 to chitosan-heparin surface was confirmed by ELISA using anti-fibronectin antibody (Sigma-Aldrich, #MAB1935, Mouse monoclonal, clone 868A11, 1 μg/ml), for 2 hr at RT; followed by incubation with anti-mouse horseradish peroxidase (HRP)-conjugated IgG secondary antibody for 1 hr at RT (1:1,000 dilution, Cell Signaling, Danvers, MA); and subsequent addition of substrate (TMB, Sigma-Aldrich Inc. St. Luis, MO) for 7 min at RT. Absorbance was read at 450 nm using a Biotek Synergy 4 Spectrophotometer (with subtraction of background absorbance of 570 nm).

SIS sheets (Cook Biotech, West Lafayette, IN, USA) were cut and sterilized by immersion in 70% ethanol and then washed with sterile PBS. Heparin was immobilized on SIS using EDC/NHS solution (20 mm EDC, Millipore Sigma, Burlington, MA, USA), 10 mm NHS (Millipore Sigma), in 50 mm MES buffer (2-ethane-sulfonic acid buffer, pH 4.5, ThermoFisher, IL, USA) overnight at room temperature. The SIS sheets were then washed with PBS, functionalized with H2R5 or H2RGD5 (1 nmol cm-2) for 4 h at 37° C. and washed prior to use.

Cell Culture and hPBMNCs Isolation

Human umbilical vein endothelial cells (HUVECs) were purchased from Lonza as a pooled donor isolation, maintained below 80% confluency in EGM2 complete media (Lonza; Allendale, NJ) in a humidified incubator with 5% CO₂ at 37° C. and used between passage 3 and 7. Human PBM-NCs were isolated from leukocyte reduction filters (LRFs) from Trima Accel (Gambro BCT, Lakewood, CO) platelet apheresis kits purchased from Roswell Park Comprehensive Cancer Center (Buffalo, NY). LRFs trap leukocytes and allow the blood product to pass through during the donation process. Removing leukocytes is important for blood product transfusion because it minimizes the risk associated with contaminating leukocytes including secretion of cytokines and histamine. In addition, LRFs were shown to be a simple method of obtaining high quality, clinical-grade human white blood cells white blood cells (WBCs).^{[35]} The kits are used to isolate platelets from prescreened, anonymous adults, and the leftover product was purchased for experimental use within 3-4 h of collection.

LRF processing was performed as follows: briefly, the outer tubing of LRF was cut and the contents allowed to flow out of the LRF via gravity into a sterile conical tube. LRFs were then washed with 10-15 mL of cold sterile 1×HBSS (Hank's balanced salt solution, Gibco) by purging the LRF via needle and syringe. Red blood cells were lysed with cold ammonium-chloride-potassium (ACK) buffer (Gibco) for 10 min followed by washing with HBSS. PBMNCs were then further isolated via layer separation on histopaque-1077 (Sigma Aldrich Inc. St. Luis, MO). The buffy coat containing PBMNCs was then washed with HBSS and kept on ice before use.

### Static Capture and Spreading of the Cells on H2R5

The surfaces of 48-well plates were prepared and functionalized with H2R5 at different concentration as discussed above. All surfaces were preblocked with 1% (w/v) bovine albumin serum (BSA) (VWR Chemicals, LLC. Solon, Ohio) in 1×PBS prior to cell seeding. HUVECs were detached with 5 mm EDTA in 1×PBS (VWR Chemicals, LLC. Solon, Ohio) and resuspended in EBM2 supplemented with 1% FBS (R&D Systems, Inc. Minneapolis, MN) and plated at 3×104 cells per well. PBMNCs were also plated at the same density and in EBM2 media on H2R5 surfaces in a humidified incubator with 5% CO2 at 37° C. Cells were allowed to bind on the surface for 1 h and then unbound cells were removed and washed with warm 1×PBS and fixed for 10 min with 4% (w/v), paraformaldehyde (PF) (Sigma-Aldrich. Inc. St. Luis, MO) at RT. Cells were then stained with Hoechst 33342 nuclear dye for 5 min (Thermo Fisher Scientific, Waltham, MA, 1:400 dilution in 1×PBS) and average of n=15 images were acquired from each well using a Zeiss Axio Observer Z1 fluorescence microscope (LSM 510; Zeiss, Oberkochen, Germany) equipped with a digital camera (ORCA-ER C4742-80; Hamamatsu, Bridgewater, NJ). Cells were counted using ImageJ software (National Institutes of Health).

For investigating the effect of blocking α4 integrin on cell binding to H2R5, PBMNCs were first pretreated with integrin α4/CD49d function blocking antibody (Novous Biologicals LLC, #BBA37, Mouse monoclonal, Clone: 2B4) in activation buffer (25 mm Tris (Sigma-Aldrich. Inc. St. Luis, MO), 2.5 mm KCl (VWR Chemicals, LLC. Solon, Ohio), 150 mm NaCl, 1 mg mL-1 BSA and 4 mm MnCl2 (Sigma-Aldrich. Inc. St. Luis, MO, pH=7.2) for 30 min at 37° C. Then 1% FBS was added and cells were plated on 1 nmol cm-2 H2R5 surface and counted as discussed above.

To investigate cell spreading on H2R5, at 1 h postseeding unbound HU-VECs were removed, the surface was washed with warm 1×PBS, media was changed to EBM2 supplemented with 2% FBS and cells were allowed to spread on H2R5. After 3 h, HUVECs were fixed in 4% PF for 10 min at RT followed by incubation in permeabilization buffer (0.1% (v/v) Triton X-100 in 1×PBS) for 10 min at 37° C. HUVECs were then blocked with blocking buffer (0.01% (v/v) Triton X-100, 5% (v/v) goat serum (ThermoFisher Scientific, Waltham, MA) in 1×PBS) at RT for 1 h. Next, samples were stained with Alexa Flour 488 Phalloidin (ThermoFisher Scientific, Waltham, MA, 1:100 dilution in 1×PBS with 1% BSA) for 2 h at RT followed by staining with Hoechst 33342 for 10 min. Images were taken and the cell area of at least n=100 cells was measured using Image.

### Flow Cytometry

After isolation, PBMNCs were plated on H2R5 surfaces and allowed to adhere for 1 hr in EBM2 at 5% CO₂ in the absence of PRP. Unbound cells were removed and the plates were washed 4-5 times with warm 1×PBS and the cells detached from the surface with using 5 mM EDTA in 1×PBS (VWR Chemicals, LLC. Solon, Ohio) and cell scraper and processed for flow cytometry or incubated in EBM2 with 0.1% PRP medium for 24 hr in an incubator (5% CO₂, 37° C.).

For flow cytometry, cells were treated with 5 mM EDTA, washed four washes with 1×PBS and mechanically detached using a cell scraper. Cells were pre-blocked in blocking buffer for 10 min followed by incubation with primary fluorescently-conjugated antibodies for 15 min on ice. The following antibodies were used: CD14-Alexa Flour® 488 (Biolegend, #301811, Mouse monoclonal, clone M5E2, 1:50 dilution), CD16-BV520™ (Biolegend, #302048, Mouse monoclonal, clone 3G8, 1:50 dilution), CD49d-PE/Cy7 (Biolegend, #304313, Mouse monoclonal, clone 9F10, 1:50 dilution) and CX3CR1-AF647© (Biolegend, #341608, Rat monoclonal, clone 2A9-1, 1:50 dilution). IgG1 isotype control was also used for proper gating. Next, cells were washed and fixed with 4% PF for 10 min on ice. Flow-cytometry was performed for 10⁴ events using a BD Fortessa X-20 (BD Biosciences) and the data was analyzed with FCS Express software suite (DeNovo Software; Naples, CA).

### Capture of Cells Under Flow

Capture of HUVEC or PBMNCs under flow was performed employing single channel PDMS device with the channel dimensions of 2 mm in width, 2 cm in length and 200 μm height. The plates were coated with 1 nM/cm² H2R5 and then were pre-blocked with 1% ESA for 1 hr at RT. Next, the device was washed with 100% ethanol, dried with air stream and sealed on the surface using binder clips. The input port was attached to the cell reservoir containing the cell suspension (5×10⁵ cell/ml) and the output port was connected to the syringe that was placed in a Harvard Apparatus Syringe Pump. HUVECs or human PBMNCs in EBM2+1% FBS were drawn into the channel at different flow rates and shear stress was calculated using following equation:

$${τ}_{ω} = \frac{6 μQ}{{h}^{2} {w}_{1}}$$

Where the μ is the viscosity, Q is the volumetric flow rate controlled by the pump, h and w₁ are height the width of the channel, respectively. At the end of the run, the channel was washed gently with 1×PBS and captured cells were fixed with 4% PF. Next, bound cells were incubated in permeabilization buffer for 10 min at 37° C. and then blocked using blocking buffer for 1 hr at RT, followed by overnight incubation at 4° C. with an antibody against human CD14 (Abgent, #AP6294A, Rabbit polyclonal, 1:50 dilution). Cells were then washed with 1×PBS and treated with rabbit Alexa Fluor 488 secondary antibody (Invitrogen, 1:200 dilution) for 1 hr at RT followed by nuclei staining by Hoechst 33342. Images were acquired with the Zeiss Axio Observer Z1 fluorescence microscope (LSM 510) and cells were counted using ImageJ software.

Platelet Adhesion Assay: Human peripheral blood was drawn from healthy donors as per University at Buffalo IRB guidelines and federal regulations by traditional method using syringe and needle and supplemented with EDTA to prevent clotting. To obtain PRP, the blood was centrifuged at 150 g for 20 min to separate RBC from PRP. The supernatant was removed carefully and this step was repeated.

Nontissue culture plates or SIS functionalized as it described above with H2R5, H2RGD5 or only heparin (negative control). PRP was placed on each surface for 60 min at 37° C. and then the surfaces were washed twice with 1×PBS. Cells on nontissue culture plates were stained for CD62 or while SIS samples were prepared for SEM imaging as follows. Samples were fixed with 2% glutaraldehyde for 2 h at 4° C., washed twice with 1×PBS, dehydrated with sequential treatments with graded ethanol (30%, 50%, 70%, 85%, 95%, and twice with 100% ethanol) for 15 min each. The ethanol was exchanged with hexamethyldisilazane and samples dried at room temperature overnight, then coated with gold and imaged using a scanning electron microscope (Hitachi SU70 FESEM, Krefeld, Germany).

### Gene Expression Analysis

For gene expression measurements, MCs were seeded at 1.5×10⁶ cells/cm² in EBM2 on H2R5 functionalized plates and after 1 hr unbound cells were removed, the surfaces were washed with 1×PBS and cells were lysed; or fresh medium was added and the cells were lysed 24 hr later. RNA was isolated and purified according to the manufacturer's protocol (Qiagen RNeasy mini kit, Qiagen; Hilden, Germany) and cDNA was synthesized using High-Capacity cDNA Reversed Transcription Kit (Thermo Fisher Scientific, Waltham, MA) following the manufacturer's protocol. qRT-PCR was performed for each sample in triplicates, using Power SYBR® Green PCR Master Mix (Thermo Fisher Scientific, Waltham, MA) as per manufacturer's instructions, using the Bio-Rad CFX96 thermal-cycler. For each condition, gene expression levels were normalized internally to the housekeeping gene RPL32, and then to the levels of fresh PBMNCs (prior to seeding). Primers were obtained from PrimerBank or the corresponding reference provided in Table 2.

TABLE 2

| Target | Forward | Reverse |
|---|---|---|
| gene | 5′ to 3′ | 5′ to 3′ |
| CD14 | GAGCTCAGAGGTTCGGAAGA | CTTCATCGTCCAGCTCACAA |
| CD16 | TTCCAGCTGTGACACCTCAG | GACAAACCTCTCCACCCTCA |
| CD68 | GGAAATGCCACGGTTCATCC | TGGGGTTCAGTACAGAGATG |
| | A | C |
| CD163 | TTTGTCAACTTGAGTCCCTT | TCCCGCTACACTTGTTTTCA |
| | CAC | C |
| IL10 | GACTTTAAGGGTTACCTGGG | TCACATGCGCCTTGATGTCT |
| | TTG | G |
| VEGFa | CACACAGGATGGCTTGAAGA | AGGGCAGAATCATCACGAAG |
| CD86 | CTGCTCATCTATACACGGTT | GGAAACGTCGTACAGTTCTG |
| | ACC | TG |
| IL12A | CCTCCACTGTGCTCCTTTTA | TCAGCAACATGCTCCAGAAG |
| TNFa | CCTCTCTCTAATCAGCCCTC | GAGGACCTGGGAGTAGATGA |
| | TG | G |
| RPL32 | GCCCAAGATCGTCAAAAAGA | TCCGCCAGTTACGCTTAATT |
| | GA | T |

### A-TEV Preparation

Each H2R5 fortified A-TEV was manufactured by wrapping SIS (Cook Biotech, West Lafayette, IN, USA) around a perforated silicon tubing (McMaster-CARR, OH, USA). The outer diameter of the tubing was 0.025″ (635 μm) and perforated manually using needles in a uniformly distributed pattern. Each SIS sheet was 50 μm in thickness and was rolled 3 times around the mandrel and dried at RT. The SIS layers were sealed together by vacuum cross-linking in EDC/NHS solution (20 mM EDC (1-ethyl-3-(3-dimethylaminopropyl) carbodiimide, Millipore Sigma, Burlington, MA, USA), 10 mM NHS (N-hydroxysuccinimide, Millipore Sigma), in 50 mM MES buffer (2-ethane-sulfonic acid buffer, pH 4.5, Thermo Fisher, IL, USA)) as shown in FIG. 11. Briefly, one side of the A-TEV mandrel was connected to the vacuum line while the other end was clamped and immersed in the crosslinking solution overnight. Vacuum facilitated penetration of EDC/NI-IS solution into the A-TEV layers through the mandrel perforations resulting in crosslinking the SIS layers together. The A-TEV was then washed with 1×PBS, sterilized by immersing in 70% ethanol, washed with PBS and allowed to dry in RT for 2 hr. Dried A-TEV was then functionalized as follows: After removing the silicone mandrel, the A-TEV was placed in EDC/NHS solution contained 4 mg/ml heparin. One side of the A-TEV was attached to the syringe pump to pass the heparin solution through the lumen overnight at RT, ensuring complete surface coverage by heparin (150 μl/hr). Heparin-coated A-TEV was then washed twice with 1×PBS and functionalized with H2R5 (250 mg/ml) with the process as heparin for 4 hr at 37° C. and washed with 1×PBS prior to implantation.

### A-TEV Implantation, Ultrasound and Removal

All animal experiments were approved by Nationwide Children's Hospital institutional guidelines for the use and care of animals (IACUC Protocol: AR20-00088). CX3CR1-confetti strain was generated by breeding of B6J.B6N(Cg)-Cx3cr1^{tm1.1(cre)Jung}^{_{/J}} (CX3CR1-Cre, Strain #025524) and STOCK Gt(ROSA)^{26Sor}^{^{tm1(CAG-Brainbow2.1)Cle}}^{/J} (R26R-Confetti, Strain #013731) that were purchased from The Jackson Laboratory (Bar Harbor, ME, USA). H2R5-based A-TEVs were implanted into the aorta of 8-10 weeks old C57BL/6 mice for 4 weeks (n=14) or 12 weeks (n=10); or CX3CR1-confetti transgenic mice for 2 weeks (n=5) or 4 weeks (n=5). After surgery, the abdomen was closed in two layers by using a 6-0 black polyamide monofilament suture, and the mice were anesthetized using ketamine/xylazine cocktail, with ketoprofen as preanesthesia analgesic. Hair in the surgical area was removed by shaving, and the area was disinfected by betadine and alcohol pads. A midline laparotomy incision from below the xyphoid to the suprapubic region was made, and a self-retaining retractor was inserted. The intestines were wrapped in saline-moistened gauze and retracted. The infrarenal aorta and inferior vena cava were bluntly defined. Microsurgery was performed using an operating microscope with zoom magnification. The aorta was separated from the inferior vena cava, and 2 microclamps were placed on both sides of the aorta, and the aorta was then transected. An aortic interposition A-TEV, 3 mm in length, was implanted with proximal and distal end-to-end anastomoses using sterile 10-0 monofilament sutures on tapered needles. Any hemorrhaging was controlled by applying topical, absorbable, sterile hemostatic agents (Surgicel). Then, intestines were returned to the abdominal cavity, and the abdominal musculature and skin were closed in two layers using 6-0 prolene sutures. Animals were moved to a recovery cage with a warming pad until the mice became fully mobile. Upon recovery, each mouse was returned to a new cage, and pain medication (ibuprofen, 30 mg kg⁻¹, drinking water) was given for 48 h. For monitoring A-TEV patency, high-frequency Doppler ultrasound were performed every two weeks (Vevo 2100; VisualSonics, Toronto, ON, Canada) after anesthetizing the mice (1.5% isoflurane; Baxter, Deerfield, IL, USA) by acquiring long-axis images in both B mode and color Doppler. At 4- and 12-weeks postimplantation, A-TEVs were explanted as follows. Mice were euthanized with a cocktail overdose of ketamine (200 mg kg⁻¹) and xylazine (20 mg kg⁻¹). Subsequently, the chest was cut open, and a small cut was made on the right atrium; mice were systemically perfused from the left ventricle with 20 mL of 0.9% saline. After perfusion, the grafts were explanted and then placed in 10% formalin overnight. A-TEVs were then dehydrated in series of graded ethanol solutions and xylene (VWR Chemicals, LLC. Solon, Ohio) and embedded in par. Next the paraffin embedded tissues were cut into 10 μm sections using a microtome (Leica Microsystems, Wetzlar, Germany) for histological analysis. For histological assessment, tissue sections were deparaffinized, rehydrated in xylene and ethanol series, and stained with Harris H&E as per manufacturer's instructions. A-TEVs explanted from CX3CR1-confetti mice at 2- and 4-weeks postimplantation, were perfused and then placed in 4% paraformaldehyde (Sigma-Aldrich. St. Luis, MO) in 1×PBS for 1 h at RT and then placed into 15% sucrose (Sigma-Aldrich St. Luis, MO) in 1×PBS for 16-24 h followed by transfer to 30% sucrose in 1×PBS overnight at 4° C. until the tissues were completely submerged. Then tissues were embedded in optimal cutting temperature (OCT) compound (Sakura Finetek USA, Inc, Torrance, CA) and frozen on dry ice. Tissue blocks were cut into 10 μm sections thick using a cryotome (Leica Microsystems, Wetzlar, Germany) and were stored at −80° C. until use.

### Immunochemistry

Following de-paraffinization with xylene and rehydration with a series of ethanol washes, tissues sections were subjected to pressure-activated high temperature antigen retrieval and immunostaining was performed as follows. Tissue sections were treated with permeabilization buffer (0.1% (v/v) triton X-100 in 1×PBS) for 10 min at 37° C. Next, tissues sections were blocked with blocking buffer (5% (v/v) goat serum in 0.01% (w/v) triton X-100/PBS) at RT for 1 hr followed by incubation at 4° C. overnight in primary antibodies diluted in blocking buffer: CD14 (Abgent Inc., #AP6294A, Rabbit polyclonal, 1:50 dilution), CD206 (Proteintech, 18704-1-AP, Rabbit polyclonal, 1:100 dilution), Ki67 (Invitrogen #PA5-19462, Rabbit polyclonal, 1:100 dilution), eNOS (BD Biosciences, #610296, Mouse monoclonal, Clone 3, 1:100 dilution), αSMA (Abcam, #ab5694, Rabbit polyclonal, 1:100 dilution), αSMA (Sigma Aldrich, A5228, Mouse monoclonal, Clone: 1A4, 1:100). Following three washes with PBS containing 0.01% triton X-100, tissue sections were incubated for 1 hr in anti-mouse/rabbit Alexa Fluor secondary antibodies (568/488, 1:200, Invitrogen). Nuclei were counterstained with Hoechst 33342 (1:400; Thermo Fisher Scientific in PBS) for 10 min at room temperature, and images were obtained with a Zeiss Axio Imager microscope (Carl Zeiss GmbH, Jena, Germany) followed by three washes.

OCT sections were washed in PBS for 10 minutes to remove OCT. Sections then treated with permeabilization buffer for 10 min in 37° C. followed by blocking with blocking buffer at RT for 1 hr and stained with CD14 as it was mentioned above. Images were taken in different Z-Stack performed by confocal microscopy, Leica Stellaris 5 (Leica Microsystems, Wetzlar, Germany)

For en-face staining imaging of the lumen, the grafts were cut open and then permeabilized, blocked and stained with Alexa Flour™ 488 Phalloidin and Hoechst 33342. The A-TEV then flattened on a glass slides and lumens were imaged using confocal microscopy (Leica Stellaris 5).

### Statistical Analysis

All data were expressed as mean±standard deviation. All experiments were repeated at least three times each with at least triplicate samples. In total n=34 H2R5-A-TEVs were implanted in mice. For confetti mice, A-TEVs were explanted at 2 weeks (n=5) and 4 weeks (n=5); for C57BL/6 mice at 4 weeks (n=14) and 12 weeks (n=10) postimplantation. Immunostaining and histology were performed from middle cross-sections of H2R5 A-TEV. Statistical significance for each experiment was determined by unpaired t-test and one-way/two-way analysis of variance followed by the Dunnet-Tukey multiple comparison test using GraphPad prism software and statistical significance were defined as p<0.05.

### Results

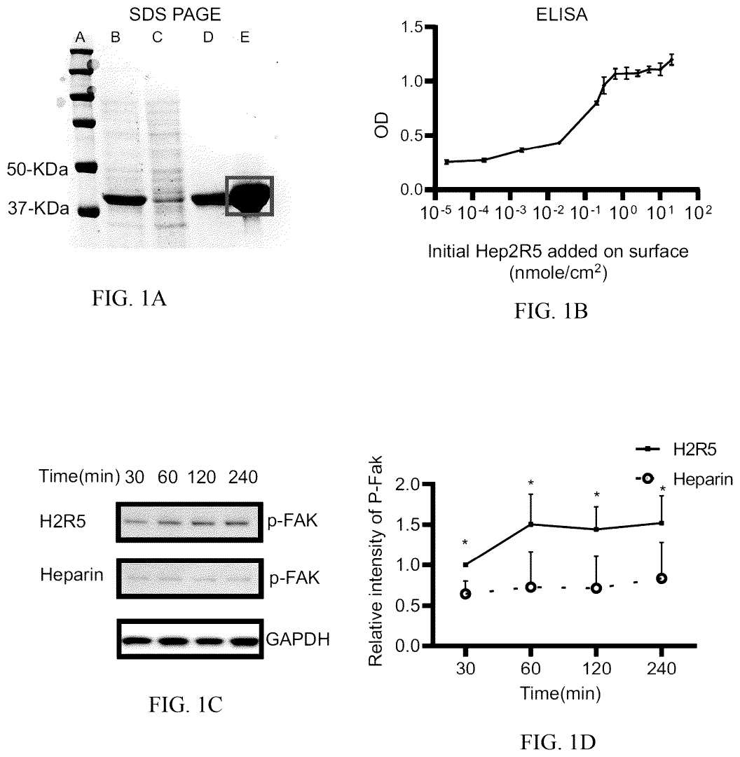

MCs are known to hover over and bind EC activated by injury or inflammation using integrin α4β1, which binds to the CS-1/CS-5 domain of the IIICS region of fibronectin as well as the immunoglobulin superfamily molecule VCAM1, which is expressed by activated endothelium. It was hypothesized that engineering a lumen mimicking the activated endothelium might attract blood MC that would turn into EC on the surface of the graft. To this end, a novel His tagged fusion protein was generated, H2R5 containing two domains: (i) the second heparin binding domain (H2) of fibronectin; (ii) five tandem repeats of the flexible linker motif, GGGS followed by a peptide from CS-5 region of fibronectin, which is known for binding specifically to the α4β1 integrin (GGGS-HIPREDVDYH) denoted as R5. H2R5 was produced in strain BL21 of E. coli after induction by IPTG and soluble protein was harvested and purified by Ni+ affinity column (See e.g., FIG. 1A).

Next H2R5 was immobilized on heparin (H) via its heparin binding domain (H2). To this end, non-tissue culture treated surfaces were coated with chitosan, followed by heparin, which binds to chitosan electrostatically. H2R5 was then immobilized on heparin and binding was confirmed by ELISA using and an antibody against the H2 domain of fibronectin, showing that the surface was saturated when the input concentration of H2R5 reached 1 nmole/cm² (See e.g., FIG. 1B).

To show that heparin immobilized H2R5 was biologically active, it was examined whether integrin α4β1 on the cell surface could bind to H2R5 and activate focal adhesion kinase (FAK), a key effector of integrin-mediated signaling. To this end, HUVEC expressing α4β1 integrin were seeded on H2R5 or heparin alone (no H2R5) and at the indicated time points, the adherent cells were lysed for protein isolation. Western blotting showed that in contrast to heparin, binding to H2R5 led to quick FAK phosphorylation within 30 minutes. FAK phosphorylation increased over time and was sustained for at least 4 hours, suggesting strong integrin engagement on the H2R5 surface (See e.g., FIG. 1C).

### Capture of HUVEC on H2R5 Functionalized Surface

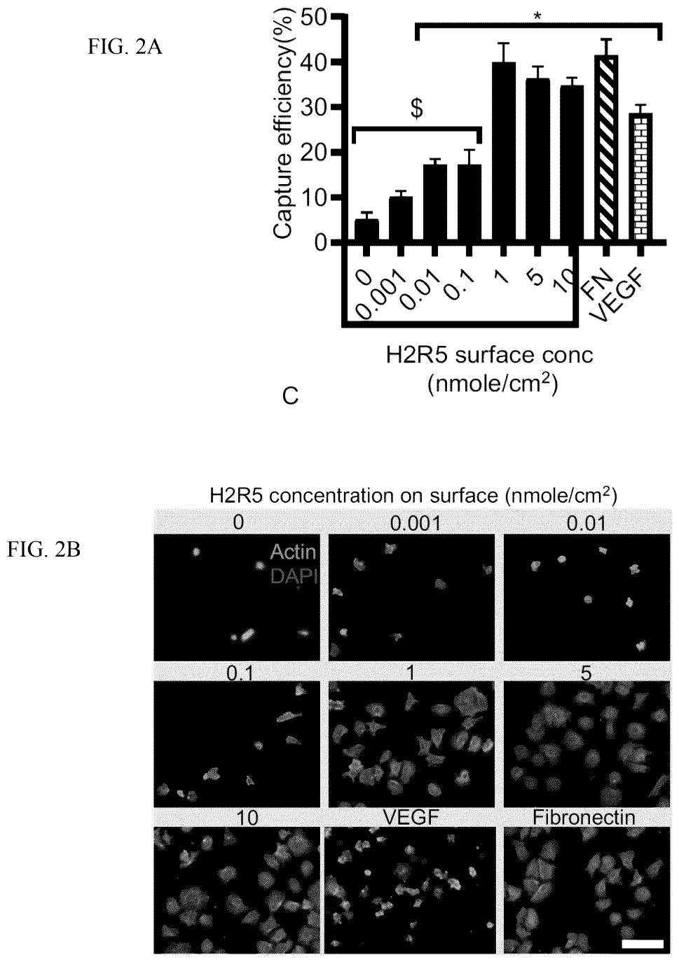

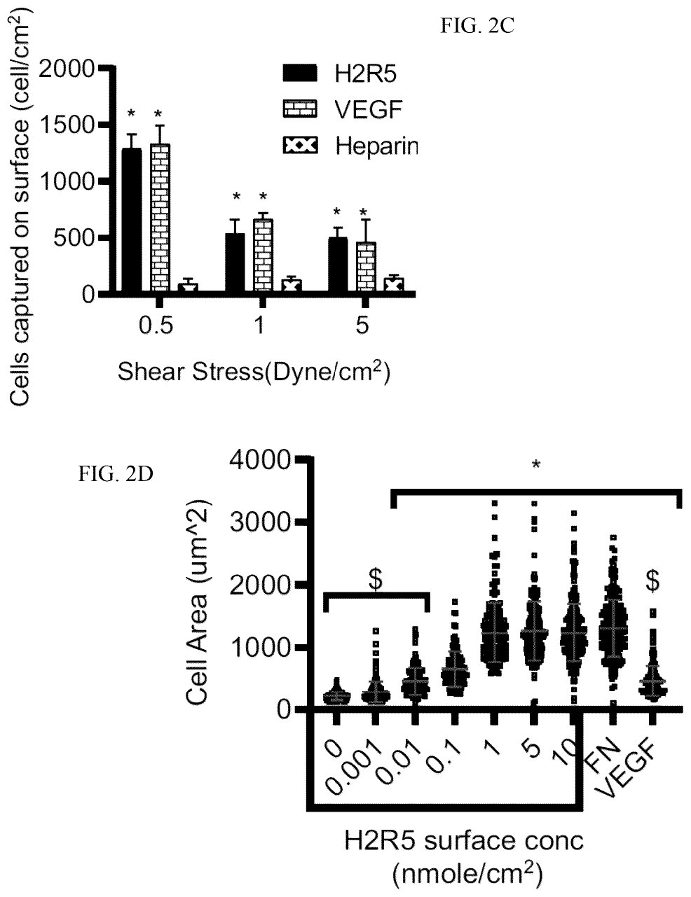

The efficiency of HUVEC capture was examined on various concentrations of immobilized H2R5. HUVEC were seeded on H2R5 surfaces in the presence of 1% serum. After 1 hr, unbound cells were washed and bound cells were stained with DAPI and counted. It was observed that the capture efficiency of HUVECs increased with increasing surface concentration of H2R5 and reached a maximum at ˜1 nmole/cm² (See e.g., FIG. 2A), the concentration that saturated the surface as shown by ELISA. To see whether binding on H2R5 could induce cell spreading, HUVECs were plated on various concentrations of H2R5 for 4 hr, stained for actin-phalloidin and cell area was measured using image J software. Similar to cell attachment, cell area increased with increasing H2R5 concentration and reached a plateau at ˜1 nmole/cm² (FIG. 2B), suggesting that binding to H2R5 led to cytoskeletal re-organization, possibly as a result of integrin engagement.

Next, to assess the ability of immobilized H2R5 to capture the cells underflow, a suspension of HUVECs was flown through a single channel (length: 2 cm, width 2 mm, height: 200 μm) microfluidic device coated with 1 nmole/cm² of H2R5, at three flow rates corresponding to shear stress of 0.5, 1 and 5 dynes/cm². As shown in FIG. 2C the number of HUVEC captured on the channel decreased with increasing shear stress from 1,293±120 cells/mm² at 0.5 dynes/cm² to 502±87 cells/cm² at 5 dynes/cm². However, even at the highest shear stress of 5 dyne/cm², the capture efficiency on H2R5 was significantly higher than that on control, heparin-coated surfaces (141±29 cells/cm²) and similar to immobilized VEGF. These results show that H2R5 could capture HUVEC under static and shear conditions, activated FAK and supported cell spreading, suggesting that it might be possible to capture α4β1 expressing monocytes (MC) from blood.

Immobilized H2R5 Captures Monocytes (MC) from Peripheral Blood Mononuclear Cells Via α4β1 Integrin

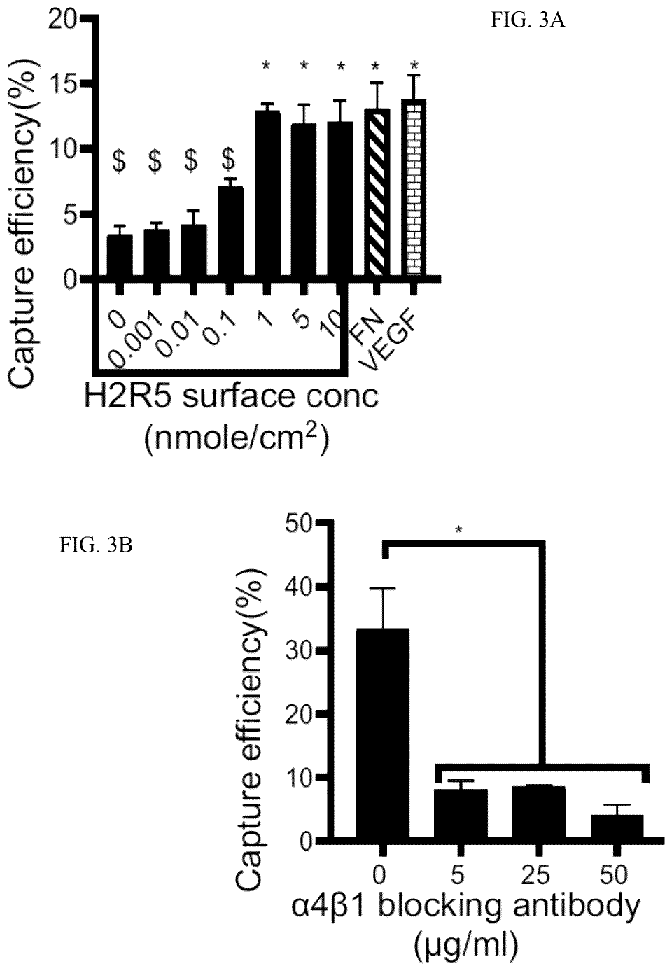

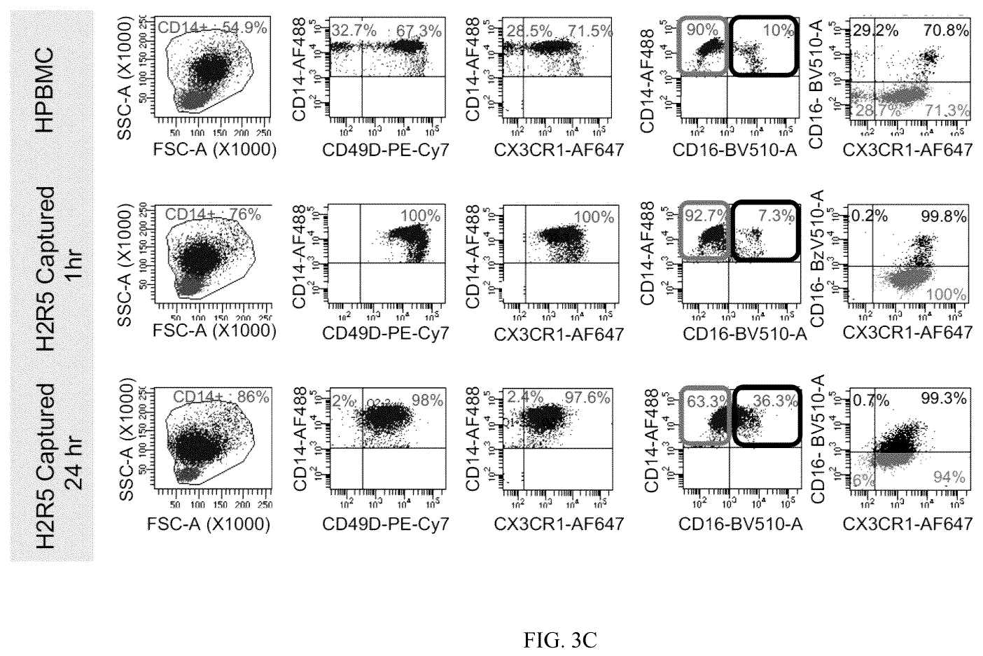

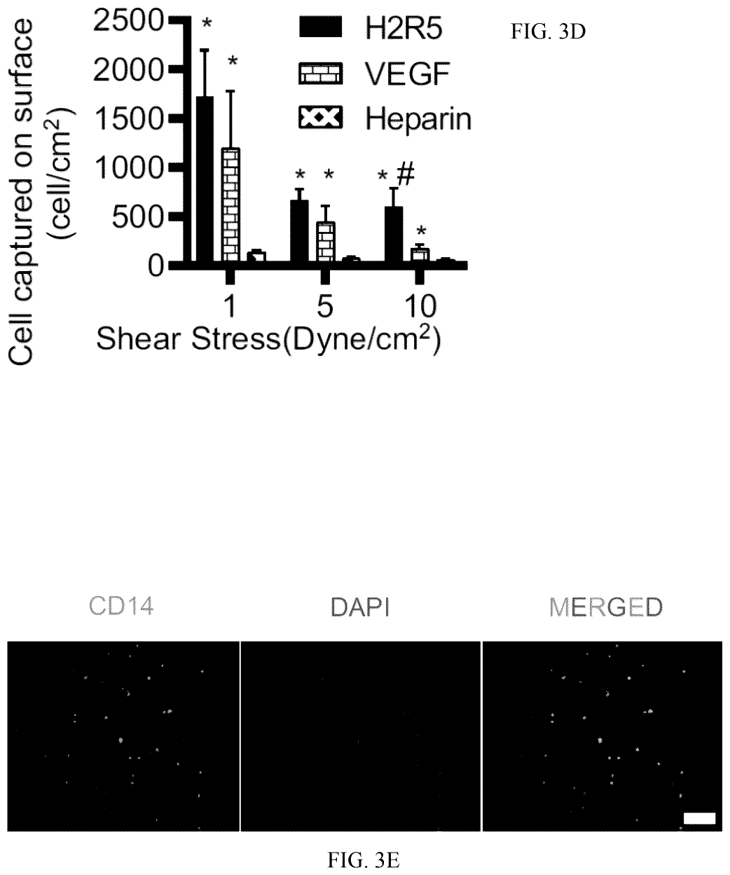

To examine the potential of immobilized H2R5 to capture MC from blood, peripheral blood mononuclear cells (PBMNC) isolated from human blood were seeded on varying H2R5 concentrations for 1 hr, unbound cells were washed and bound cells were counted. Similar to HUVEC, the number of captured PBMNC increased with increasing H2R5 surface concentration and reached a maximum when the surface was saturated with H2R5 (FIG. 3A). Pre-treatment of PBMNC with a function-blocking antibody against α4β1 integrin prevented capture onto the H2R5 surface (FIG. 3B) demonstrating that PBMNC bound to H2R5 via α4β1 integrin.

Next, the percentage of PBMNC that bound to H2R5 were MC was examined. To this end, PBMNC were allowed to adhere for 1 hr and then detached by using 5 mM EDTA in PBS and gentle mechanical removal sing cell scraper, processed for multi-color flow cytometry. As indicated in FIG. 3C, while ˜55% of total PBMNC were CD14+, the percentage of H2R5-bound cells that were CD14+ increased to 76% after 1 hr suggesting preferential capture of MCs on H2R5. The % CD14+ cells increased further to 86% after 24 hr, indicating MC enrichment over time on the H2R5 surface. While 67% and 71% of CD14+ PBMNCs were positive for CX3CR1 and CD49d (α4β1 integrin), all H2R5 bound CD14+MC were positive for CX3CR1 and not surprisingly, CD49d after 1 hr of capture. Like PBMNC, H2R5 captured cells were highly enriched in classical MC (CD14++/CD16−: 92.7% vs. non-classical CD14+/CD16+: 7.3%), however they became enriched in CD16+MC after 24 hr (CD14++/CD16−, 63.3% of MC+ vs. non-classical CD14+/CD16+, 36.3% of MC). Surprisingly, whereas ˜70% of CD16+ cells in PBMNCs expressed CX3CR1, 99.8% of CD16+ cells that were captured on H2R5 were positive for CX3CR1, suggesting the preferential capture of CX3CR1+ cells known as patrolling MCs on H2R5 surface (FIG. 3C).

To determine the potential of H2R5 to capture MC under shear, a suspension of PBMNC was flown in a microfluidic channel under different shear stress and then fixed and stained for CD14. As expected, the number of cells bound to H2R5 decreased with increasing shear stress from 1,721±473 cells/mm² at 1 dyne/cm² to 597±191 cells/cm² at 10 dynes/cm², but the number of captured cells was significantly higher than cells captured on the heparin only surface at all shear stresses and the VEGF-coated surface at the highest shear stress (172±43 cell/cm²) (FIG. 3D). In addition, immunostaining showed that 72±6% of H2R5-captured cells under shear were CD14+, indicating MC phenotype (FIG. 3E).

To examine the effect of H2R5 functionalization on platelet recruitment, H2R5 or heparin functionalized surfaces—nontissue culture plastic (FIG. S1A) or SIS (FIG. S1B) were treated with plasma obtained from peripheral blood for 60 min. As control, we generated another fusion protein, H2RGD5 containing the H2, second heparin binding domain of FN and five tandem repeats of the flexible linker motif, GGGS followed by the well-known RGD peptide from the cell binding domain of FN, which is known for binding specifically to the α5β1 integrin (GGGS-GRGDS). After incubation with blood plasma, the surfaces were then washed, fixed, and either stained with CD62 or prepared for scanning electron microscopy (SEM) imaging (FIGS. S1A and S1B). The number of platelets bound to H2R5 (701.2±409.5 platelets mm⁻²) was much smaller than the number bound to H2RGD5 (11040.3±1586.5 platelets mm-2, p<0.005). In addition, the H2R5 surface contained mostly single platelets but the H2RGD5 contained multiple cell aggregates and therefore, the number represents a lower bound estimate. As expected, only a very small number of platelets bound to the heparin surface (11.6±4 platelets mm⁻²). This result shows that in addition to attracting MC, H2R5 is superior to H2RGD5 in preventing platelet adhesion and therefore, an appropriate choice for engineering vascular grafts.

### H2R5 Bound MC Express Higher Levels of M2 Macrophage Genes

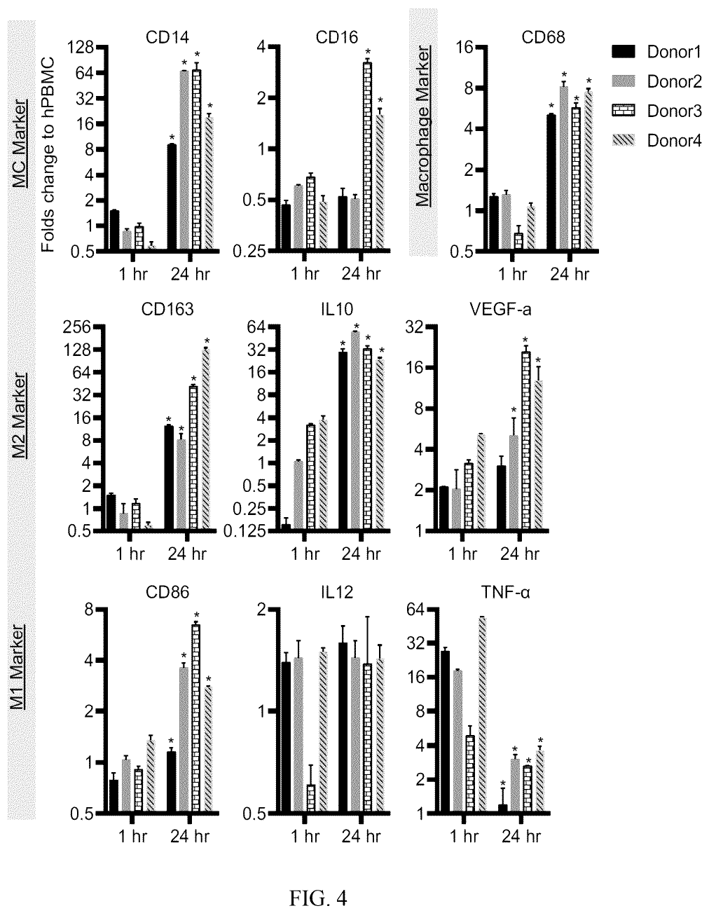

Next, the effect of H2R5 binding on macrophage polarization was investigated (FIG. 4). PBMNC isolated from blood were seeded on H2R5 surfaces. Unbound cells were washed after 1 hr and bound cells remained onto H2R5 for 24 hr in the presence of 1% human platelet rich plasma (PRP). Real time RT-PCR showed that although H2R5 bound MC expressed both M1 and M2 genes at 24 hr post seeding, M2 genes were expressed to a much higher extent as compared to total PBMNC. Specifically, CD163 and IL10 expression increased significantly by 47- and 35-fold (average from n=4 donors), while CD16 showed a more modest 4-fold increase in two of the four donors but remained unchanged in the other two. Results from two additional donors also showed increased CD16 expression (Donor 5: 1.25- to 4.8-fold; Donor 6: 0.7- to 1.1-fold). Except one donor, for which VEGFA expression remained relatively unchanged, cells from the other three donors showed 12-fold increase in VEGF expression (average from n=3 donors). On the other hand, M1 markers like CD86 increased by 3.5-fold; IL12 didn't change significantly; and TNF-α decreased significantly by 90% from 26- to 2.6-fold (average from n=4 donors). These results suggested that binding of peripheral blood MC to H2R5 might favor polarization towards the M2 phenotype.

### H2R5 A-TEV Implantation, Patency and Re-Cellularization

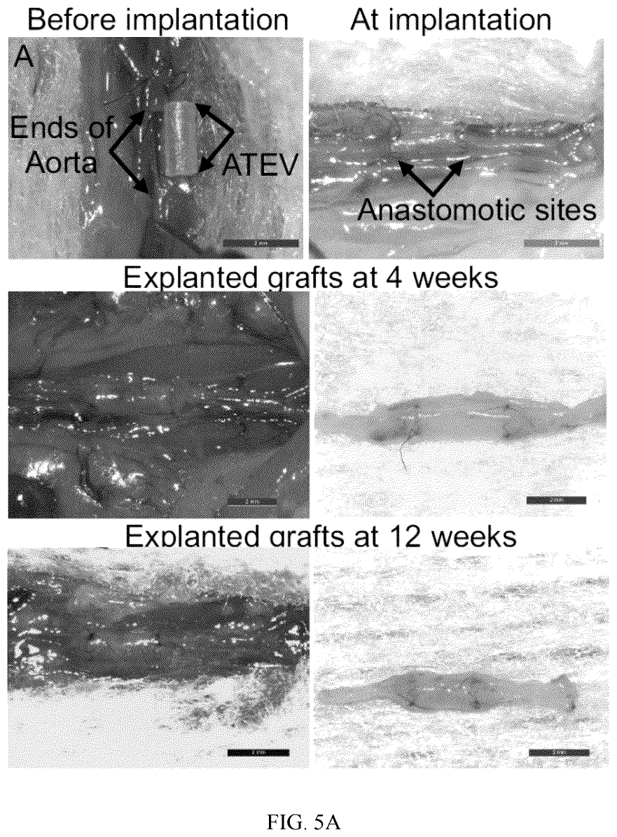

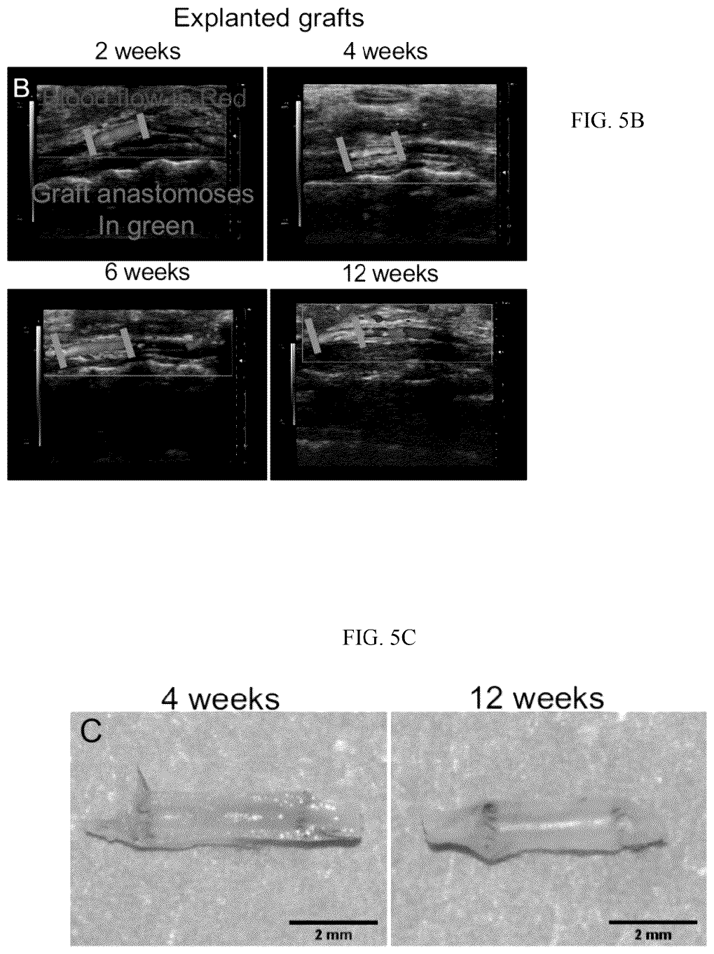

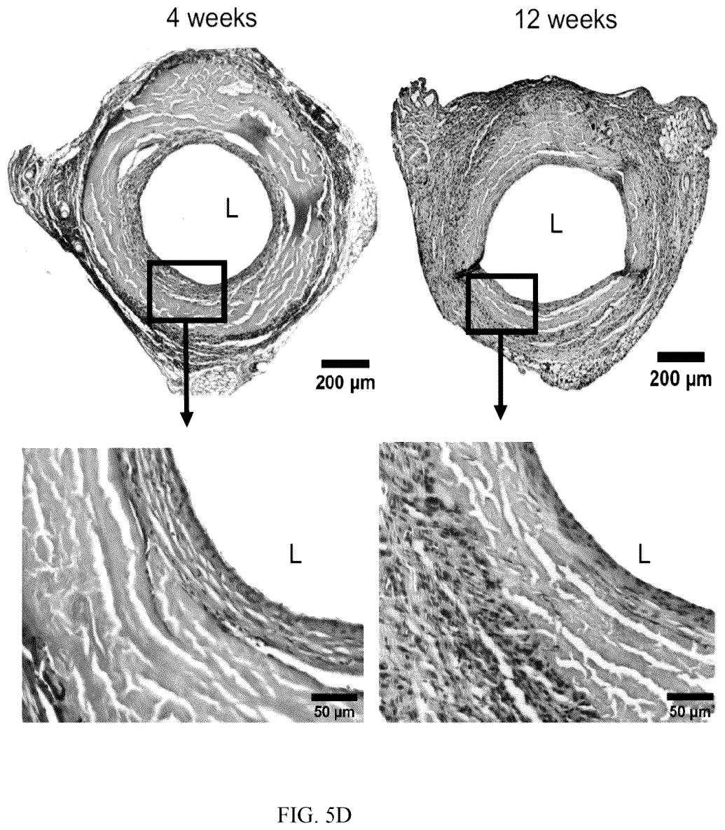

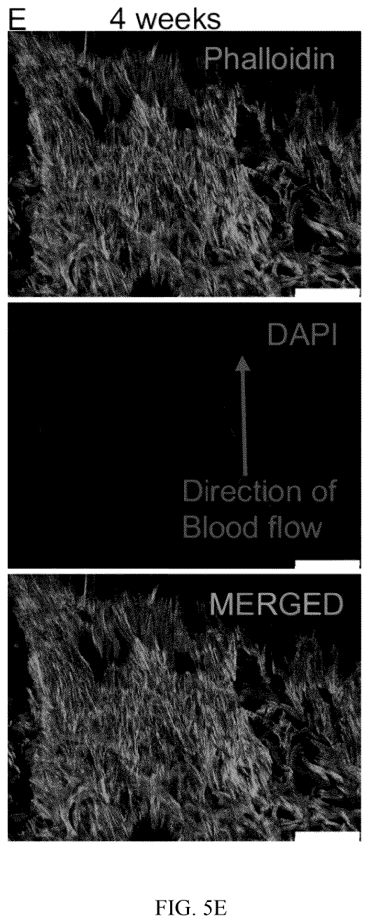

The capacity of H2R5 to bind peripheral blood MC under flow suggested that H2R5-fortified vascular grafts might be able to attract MC to the lumen, promoting endothelialization and patency, even in ultrasmall diameter grafts. To examine this hypothesis, we prepared SIS-based A-TEVs with very small inner diameter (600 μm) and wall thickness that was similar to native (150 μm) using perforated silicone tubing with 600 μm outer diameter. After rolling, SIS was vacuum pressed, cross-linked with (1-ethyl-3-(3-dimethylaminopropyl) carbodiimide (EDC)-N-hydroxysuccinimide (NHS) and then functionalized sequentially with heparin and H2R5 (FIG. S2). H2R5-functionalized A-TEVs (length: 3 mm) were implanted interpositionally into the infrarenal aorta of adult mice. All grafts were fully integrated to native tissue and explanted at 4 weeks (n=14) or 12 weeks post implantation (n=10) for further analysis (FIG. 5A).

All grafts remained patent over the course of the study as evidenced by two-color Doppler ultrasound that was performed bi-weekly (FIG. 5B). In addition, the lumens of explanted A-TEVs were clean with no signs of thrombosis (FIG. 5C). Hematoxylin and eosin (H&E) staining of entire ring sections from 4- and 12-week explanted A-TEV showed host cells infiltration onto the lumen and the layer underlying SIS (FIG. 5D). The inner diameter was similar throughout the graft length (471.7±11.6 μm at 4 weeks, and 512.2±31.2 μm at 12 weeks postimplantation, p<0.05), indicating no sign of dilation or stenosis throughout the graft. Interestingly, the acellular middle layer was present within the graft, even after 12 weeks in the animal, as the SIS scaffold degraded slowly overtime reaching 82.8±2.82% (thickness 124.2±4.24 μm) of the initial SIS by 4 weeks and 64.1±13.3% (thickness 96.6±20.0 μm, p<0.05 as compared to 4 weeks) by 12 weeks postimplantation (FIG. 5D). The luminal layer thickness remained similar throughout the study, suggesting no intimal hyperplasia (54.12±10.1 μm at 4 weeks; and 37.43±14.77 μm at 12 weeks postimplantation, p>0.05). Enface images of the lumen stained with actin-phalloidin and DAPI showed that by 4 weeks the lumen contained a confluent cell layer and cells were aligned in the direction of blood flow (FIG. 5E, red arrow).

The Lumen and Vascular Wall were Populated with Cells Co-Expressing Vascular and MC/MΦ Markers

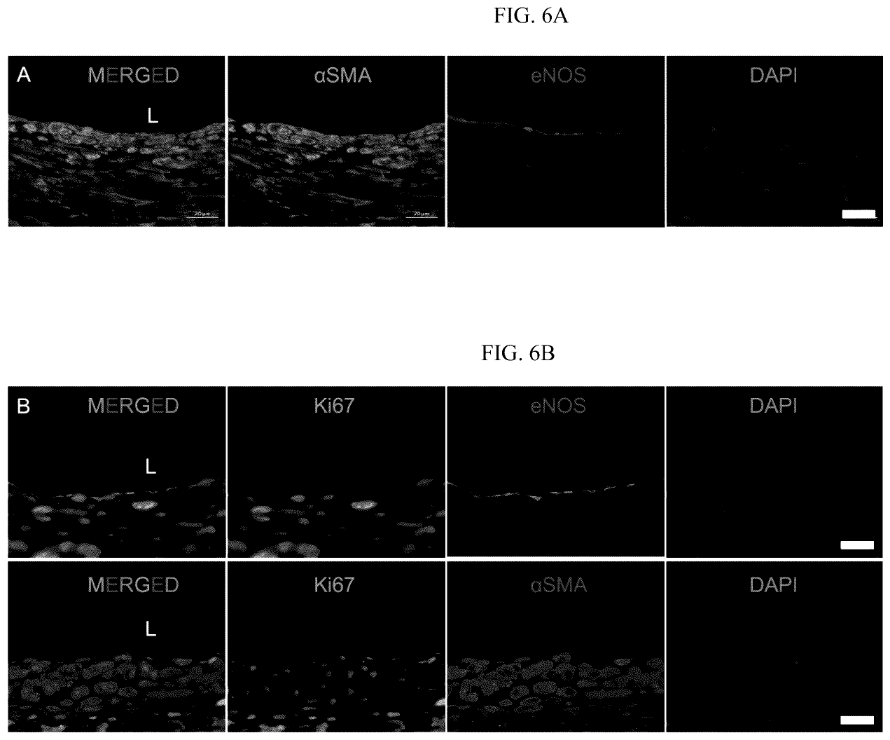

Immunostaining of explanted A-TEV sections confirmed the presence of continuous endothelial monolayer expressing eNOS (100%, n=150 lumen cells) with several underlying cells layers expressing αSMA (56.2±21.3%, n=750 cells at 4 weeks, 74.2±12.4%, n=600 at 12 weeks, p<0.005) (4 weeks, FIG. 6A; 12 weeks, FIG. S3A). Interestingly, a significant fraction of cells that populated the A-TEVs were positive for Ki67 (42.4±16.9% at 4 weeks, 40.1±15% at 12 weeks, n=450 total cells, p=0.77), indicating robust proliferation of cells in A-TEV postimplantation (4 weeks, FIGS. 6B and 6C; 12 weeks, FIGS. S3B and S3C). Cells below the top luminal layer coexpressed αSMA and myosin heavy chain (MYH11), indicating formation of an SMC containing medial layer (FIG. S4).

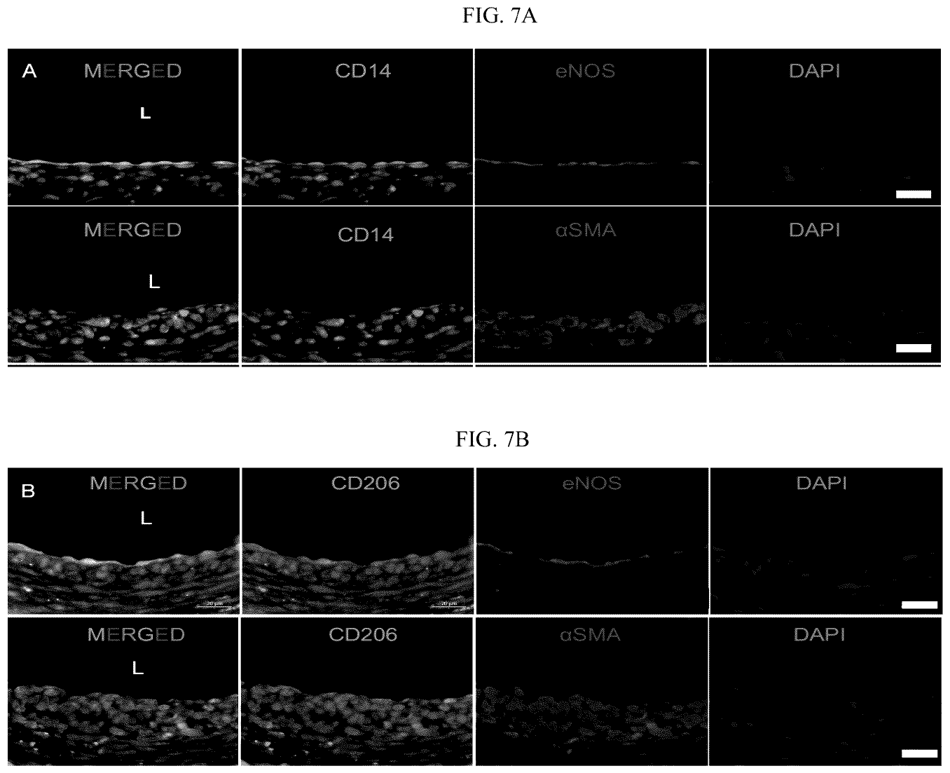

Notably, all cells in the top lumen layer and the vast majority of cells in the underlying layers stained positive for CD14 (92.91±9%, n=300 medial layer cells at 4 weeks; 95±3.2%, n=300 medial layer cells at 12 weeks, p=0.057). On the top of the lumen, all CD14+ were also eNOS+, while in the underlying layers a large fraction of CD14+ cells were also αSMA+ (65.4±24.1%, n=150 at 4 weeks; 88±5.3%, n=125 for 12 weeks, p=0.11) (FIG. 7A; FIG. S5A). The vast majority of cells also expressed the M2 macrophage marker CD206 (lumen: 93.3±11.0% at 4 weeks, 98.2±4.5% at 12 weeks, n=60 lumen cells, p=0.28; medial layer: 88.3±10.8% at 4 weeks, 95.4±3.1% at 12 weeks, n=400 media layer cells, p=0.14). All cells on the top lumen layer coexpressed CD206 and eNOS, while the majority of underlying cells coexpressed CD206 and αSMA (78.3±20.0% at 4 weeks; 73.6±9.0% at 12 weeks, n=150, p=0.75) (FIG. 7B and FIG. S5B).

A-TEV Transplantation into Confetti Mice to Trace MC/MΦ Populating the Grafts

To trace MC/MΦ cells that populate the H2R5 ATEV, a monocyte specific confetti mouse was generated to enable monitoring of cells and their progeny populating the grafts over time in a process that is known as clonal lineage tracing. To this end, the CX3CR1-Cre was crossed with R26R-Confetti to generate CX3CR1-Confetti mice. R26R-Confetti contains a strong constitutive CAG promoter followed by a floxed-STOP cassette and the Brainbow 2.1 cassette inserted in Gt(ROSA)26Sor locus. The Brainbow 2.1 region contains two loxP-flanked cassettes, each containing two fluorescent reporters positioned in head-to-tail orientation. One cassette contains nuclear-localized green fluorescent protein (nGFP) and a reverse-oriented cytoplasmic yellow fluorescent protein (YFP). The other contains the cytoplasmic red fluorescent protein (RFP) and a reverse-oriented membrane-tethered cyan fluorescent protein (mCFP). In CX3CR1-Confetti mice, Cre is expressed under the CX3CR1 promoter, which is active mostly in monocytes, dendritic cells, NK cells and microglia cells. Depending on the LoxP sites that Cre acts upon, one of the four possible fluorescent proteins, or no fluorescent protein is expressed in a stochastic manner.

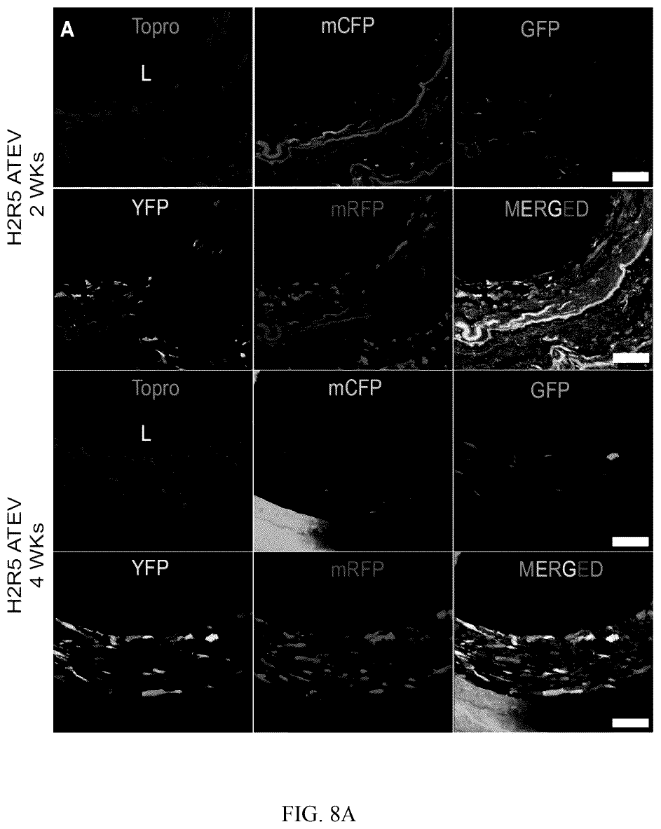

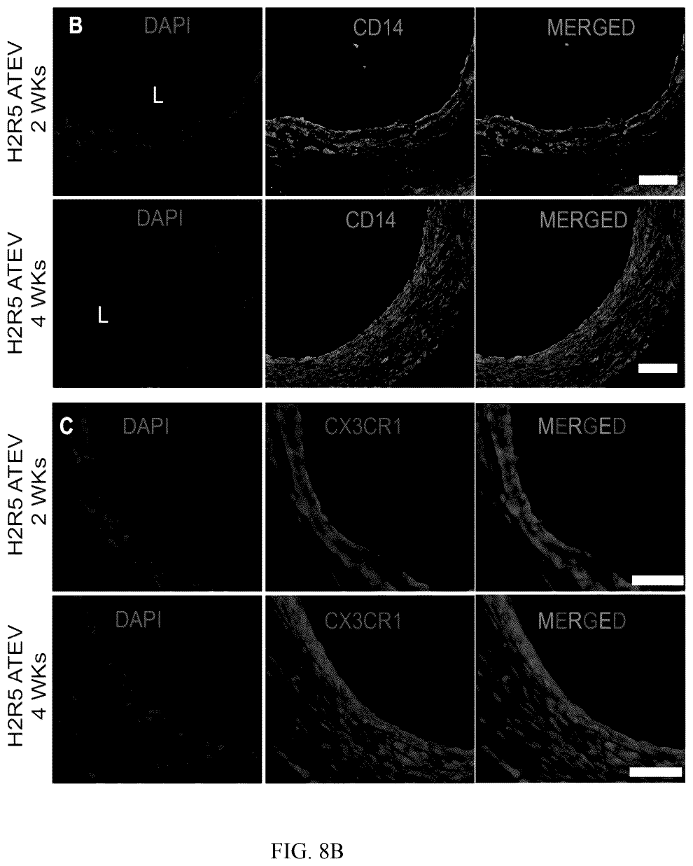

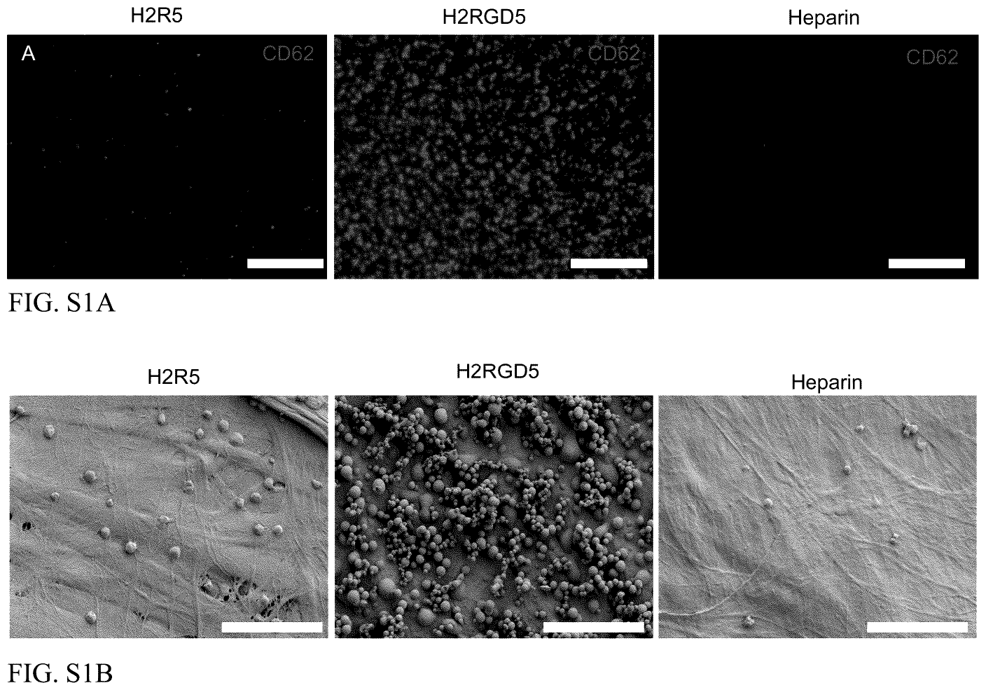

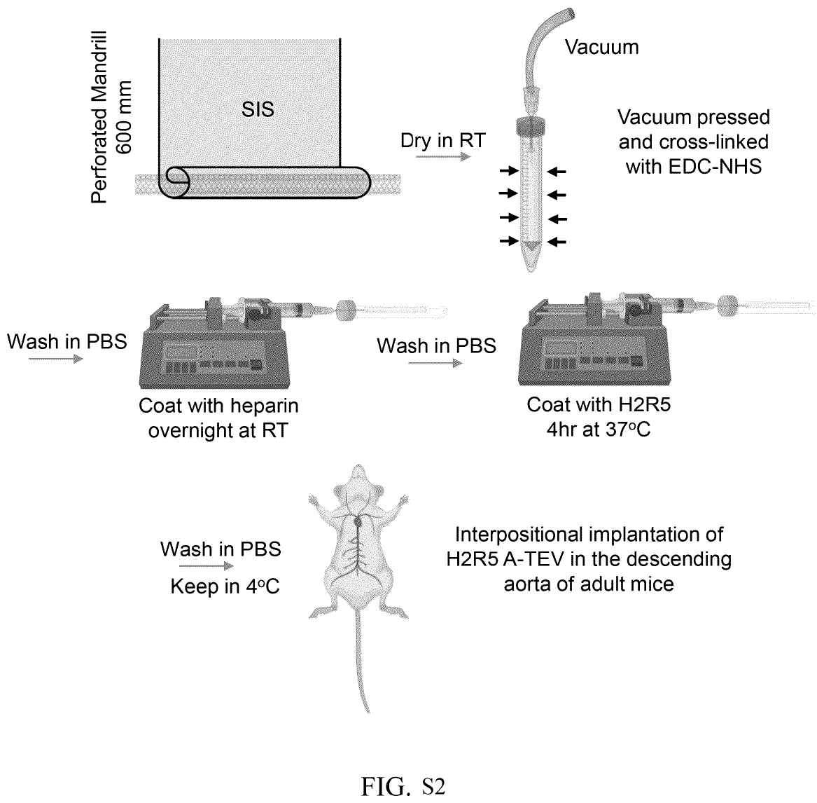

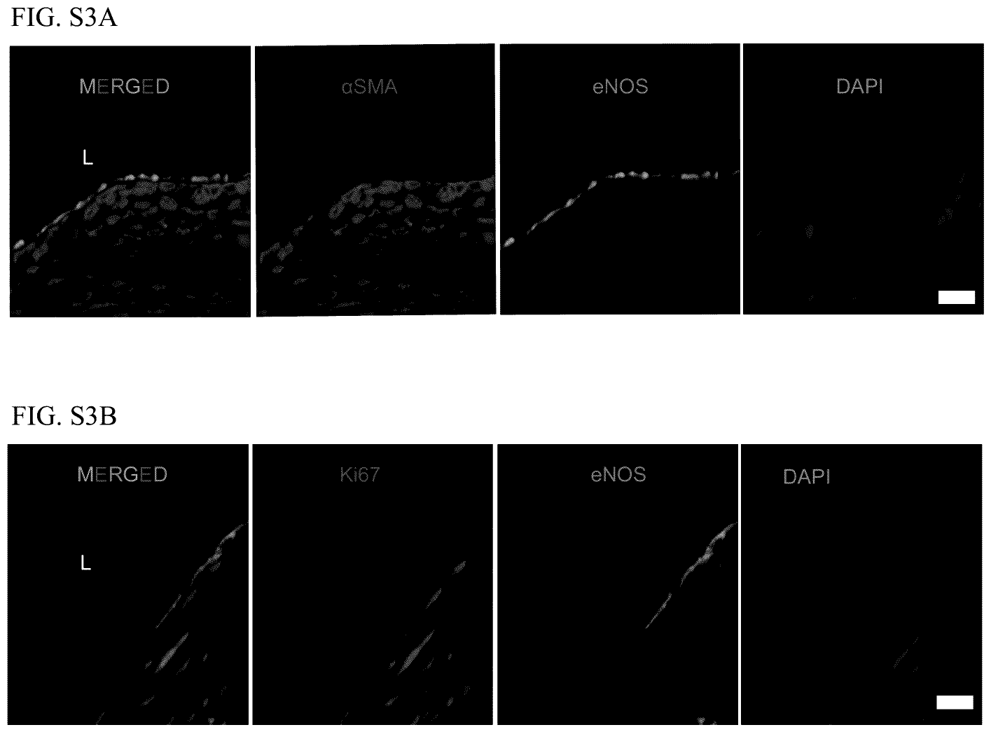

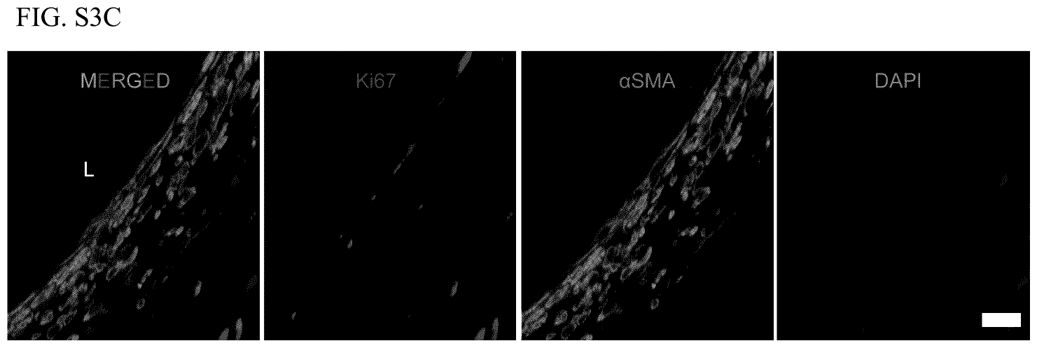

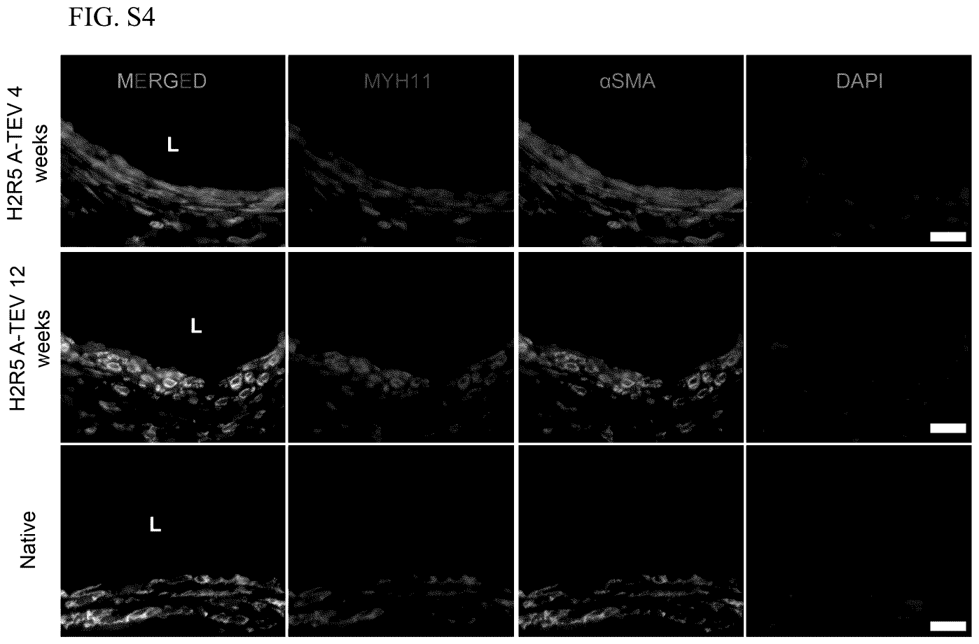

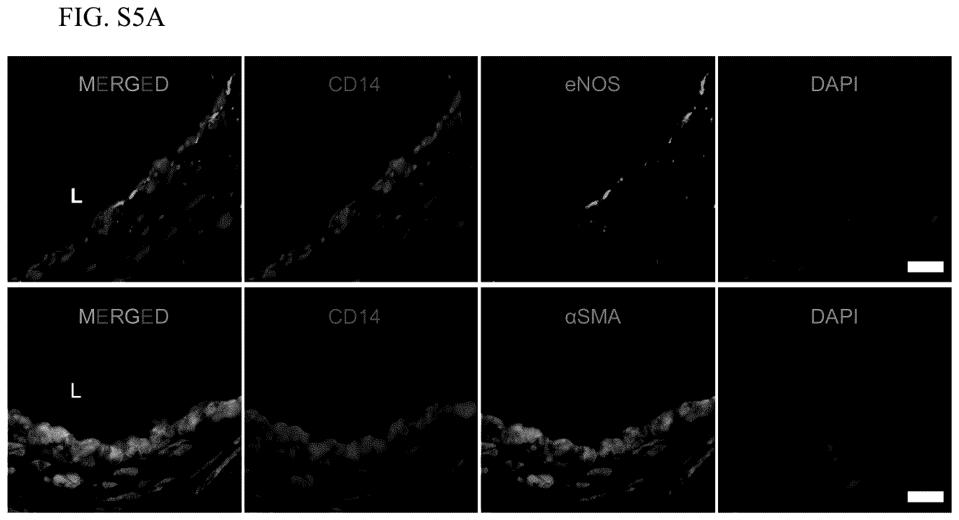

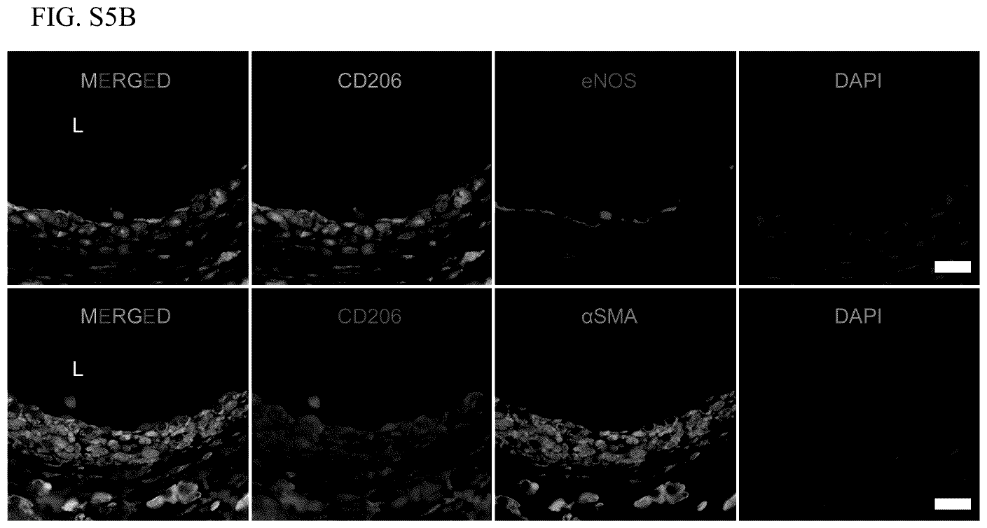

A-TEVs were implanted into the infrarenal abdominal aorta of the CX3CR1-Confetti mice as vascular interposition grafts and the grafts were harvested and evaluated by confocal microscopy at two and four weeks post-implantation. Imaging of confetti tissues showed the presence of positive fluorescent cells by 2 weeks (15.9±9% YFP+, 53±14% RFP+, 0.3±0.45% mCFP+, n=900 total cells) (10.45±6.8% nGFP+ overlapped with the YFP+ cells and could not be differentiated from them). By 4 weeks post implantation, the distribution changed slightly with an increase in mCFP+ cells (11.7±1.6% YFP+, 49.8±30% RFP+, similar to the 2-week data, p>0.05; while mCFP+ cells increased significantly as compared to 2 weeks to 3±2.4%, p<0.05; n=2300 total cells) (7.3±0.65% nGFP+ cells overlapped with the YFP+ cells and could not be differentiated from them) (FIG. 8A). Immunostaining showed that the majority of cells expressed CX3CR1 with the cells on the top layers expressing higher level than the cells in the underlying layers (FIG. 8B). In addition, all cells stained positive for CD14, indicating MC/MΦ identity (FIG. 8C). These results showed that MC from peripheral blood could be captured on the H2R5-fortified A-TEV lumen, and eventually differentiate into EC or SMC that continued to express macrophage proteins several weeks post-implantation.

### Discussion

MC and macrophages (Mφ) play a crucial role in vascular repair and remodeling via secretion of various cytokines, growth factors, and ECM remodeling enzymes. MC/Mφ trafficking to tumors enhanced angiogenesis and tumor ingrowth, while α4β1 antagonists suppressed MC/Mφ attachment to the CS-1 domain of fibronectin and VCAM1, reducing blood vessel density by 50% and tumor growth by 25%. Recruitment of host MC/Mφ mediated tissue engineered vascular graft regeneration, while depletion of macrophages by clodronate prevented neovessel formation/vascular repair in implanted tissue engineered grafts and balloon injured arteries. In a recent study from our laboratory, we reported a novel mechanism of A-TEV endothelization via MC/Mφ that were recruited to the graft lumen, differentiated into a mixed EC/Mφ phenotype and further developed into mature arterial EC under high shear stress, developing VE-cadherin junctions and producing nitric oxide that maintained A-TEV patency. In these studies, MCs were recruited successfully on the lumen of grafts containing immobilized VEGF in both mice and sheep animal models. While VEGF is an angiogenic growth factor, we reasoned that a cell adhesion promoting protein, such as H2R5 might be better suited for capturing MC under flow in the high shear stress environment of the artery. Indeed, we found that at higher shear stress (10 dyn cm⁻²), MC capture was significantly higher on H2R5 as compared VEGF surfaces. Given the abundance of circulating MC in the blood and their ability to interact with EC via integrin α4β1, we hypothesized that engineering a surface that mimics the injured/inflamed endothelium might be effective in recruiting MC to the graft lumen under shear, and that the newly recruited MC would differentiate into EC and promote arterial endothelialization and patency.

To this end, we developed a strategy to recruit monocytes from peripheral blood to cell-free vascular grafts using a novel fusion protein, H2R5 that was immobilized on the lumen and graft surface. SIS-based A-TEVs that were decorated with H2R5 formed a confluent endothelium within 4 weeks postimplantation and remained patent. H2R5 was engineered with five REDV repeats to enhance the cell binding affinity to the graft lumen, as multivalent REDV peptides were shown superior ability to bind EC and promoting angiogenesis as compared to monovalent REDV. REDV is known to be the ligand for integrin α4β1, which is only present on MCs, ECs, and leukocytes but not on platelets, SMC or fibroblasts thereby avoiding thrombosis or hyperplastic tissue ingrowth. Indeed, our results demonstrated that in contrast to H2RGD5, which promoted platelet adhesion and aggregation, H2R5 prevented platelet attachment, indicating antithrombogenic properties. The R5 region was fused to the H2 of FN to enable immobilization onto heparin, which is also known for its antithrombotic properties.

While the REDV peptide has been used widely and shown to promote adhesion, migration, proliferation of ECs and EPCs and promote angiogenesis, no studies have shown that the multivalent peptide could capture circulating MC from blood and promote endothelialization of implanted medical devices by recruited MCs. Our findings showed that H2R5 captured cells from PBMNC in a surface concentration dependent manner, reaching the highest cell density when the surface was saturated with H2R5. Conversely, pretreatment of PBMNC with a function-blocking antibody reduced the capture efficiency by ≈75%, and cell capture on control heparin surface was significantly lower, indicating that cell capture was mediated via H2R5 binding to integrin α4β1. Interestingly, while α4β1 is expressed also on lymphocytes, T-cells, and B-cells, MCs were the prevalent cell type captured on H2R5 and were further enriched 24 h after the initial attachment. This result suggests that MC might have outcompeted other blood cells for binding to H2R5. It could also reflect the fact that neither T nor B cells are adherent, whereas MC differentiate to macrophages within 24 h and adhere. In agreement, previous results showed that while VCAM1 on the surface of EC supported adhesion of both MCs and lymphocytes through integrin α4β1, binding of MC to EC under flow was four times greater than that of T cells. Stimulation with chemokine and chemoattractants further increased α4 integrin affinity preferentially in MCs rather that lymphocytes resulting in even higher MC binding to immobilized VCAM1.

MCs that bound to the H2R5 surface were comprised of a combination of classical and nonclassical phenotypes. Classical MCs (CD14⁺CD16⁻), also known as pro-inflammatory MCs are the first responders recruited to the injured endothelium and differentiate toward inflammatory macrophages. Intermediate (CD14⁺CD16⁺) and nonclassical (CD14⁺CD16⁺⁺) monocytes, also known as anti-inflammatory/patrolling monocytes express high levels of CX3CR1 and are less prevalent in the blood. This population is recruited to the injury site later and has proregenerative capacity, supporting matrix remodeling, angiogenesis, and arteriogenesis. Interestingly, loss of CX3CR1 resulted in reduced recruitment of nonclassical MC to injured carotid artery sites and impaired arterial regeneration in transgenic mice. Other studies showed that both M1 and M2 macrophages were required for angiogenesis and absence of either M1 or M2 cells resulted in impaired vascularization of bio-engineered scaffolds. Our flow cytometry results showed that all MC bound to H2R5 were CD49d⁺ and CX3CR1⁺ similar to EC patrolling MCs. Interestingly, H2R5 captured MCs with similar composition (CD14⁺ vs CD16⁺) as those in blood but captured cells upregulated CD16 after 24 h, suggesting that binding to the H2R5 surface might have promoted a nonclassical, anti-inflammatory macrophage phenotype. Indeed, while H2R5-captured MC upregulated M1 genes moderately, e.g., CD86, they upregulated M2 genes, e.g., as CD163 and IL10 to a much higher extent; and they downregulated pro-inflammatory genes such TNF-α, suggesting that binding to H2R5 might favor polarization toward M2 macrophages, which may be important for endothelialization of vascular grafts.

Previous studies described that subpopulation of circulating CD14⁺ PBMNCs that were obtained by binding to FN and termed monocyte-derived multipotential cells (MOMCs) could be a source of vascular progenitor cells and gave rise to ECs. Others suggested that blood EPC might be originating from CD14⁺/CD34low PBMNC that may be the source of MOMC. By contrast, another study reported that CD14 MCs failed to differentiate to EC, but it is worth mentioning that the culture conditions between these studies were different. While the former studies reported that MOMC generation requires soluble factors from CD14⁻ suspended cells for the first 3 days in culture, in the latter, the authors removed the unbound cells 24 h postseeding, thereby eliminating any soluble factors that they might secrete. More recently, our group demonstrated that human MCs can turn into ECs on the luminal surface of vascular grafts that were implanted into the arterial environment of mouse and sheep animal models.

In this study, we demonstrated that MCs were recruited to the lumen of vascular grafts and turned into vascular cells using a ligand of integrin α4β1. All cells on the graft lumen of H2R5-decorated A-TEVs stained positive for CD14, supporting their MC/Mφ identity. In addition, H2R5 grafts demonstrated significant tissue regeneration 4 weeks postimplantation as evidenced by the presence of a functional EC monolayer expressing eNOS and a medial layer consisting of αSMA+/MYH11+ cells. Costaining of ECs and SMCs with MC and Mφ markers, CD14 and CD206, showed that the captured cells coexpressed MC/Mφ proteins as they differentiated into vascular EC or SMC. This is in agreement with our previous work describing implantation of VEGF-decorated A-TEV in the carotid artery of sheep, where host cells populating the graft lumen coexpressed EC (CD144, eNOS) and MC/Mφ (CD14, CD163) markers at 1- and 3-months postimplantation. These experiments provide strong evidence that EC in the lumen may be originating from blood MC that are bound to H2R5 on the graft surface and develop an EC phenotype, while maintaining aspects of their original identity.

To obtain direct evidence on the origin of the host cells populating the vascular grafts, we also performed lineage tracing experiments by implanting H2R5-decorated A-TEVs into the abdominal aorta of CX3CR1-confetti mice. At 3 days postimplantation, most luminal cells were RFP+ but the number of captured cells was to low to assess statistical significance at this early time point. At 2- and 4-weeks postimplantation, the lumen consisted of multiple cell layers with the majority of cells expressing RFP or YFP, indicating their origin from CX3CR1-expressing MC. However, a significant fraction of cells (≈30-35%) did not express a fluorescent reporter, but did express both CX3CR1 and CD14 proteins as shown by immunostaining. This discrepancy could be explained by the stochastic nature of gene expression in the confetti system. Specifically, depending on the LoxP sites that Cre acts upon, cells may express one of the four fluorescent proteins or no fluorescence protein at all. The latter occurs when recombination outcomes do not remove the STOP cassette, thereby preventing gene expression. In addition, low CX3CR1 promoter activity might lead to low levels of Cre recombinase, which was reported to be associated with low probability of removal of the LoxP-STOP-LoxP region and therefore, fewer fluorescent cells. Indeed, it was reported that while CX3CR1 was expressed at high levels in circulating MCs, its expression decreased upon MC differentiation into macrophages. Similarly, GFP expression decreased in bone marrow derived MCs from CX3CR1GFP mice upon differentiation to macrophages in vitro and in vivo. In agreement, our flow cytometry results showed decreased CX3CR1 expression 24 h after capture of MC onto the H2R5 surface (mean fluorescence intensity, PBMNC: 11411.3±3284.9 vs MC at 24 h postseeding: 2563±2277.8, n=3, p=0.016). Taken together, the stochastic nature of Cre-mediated re-combination events, which may also be affected by the level of CX3CR1 promoter activity, could explain why some cells on graft do not express any fluorescent reporter.

In summary, our results indicate that immobilized H2R5 enabled capture of circulating MC from blood on the lumen of A-TEV via α₄β₁ integrin. MCs were then polarized to a mixture of M1/M2 macrophages and differentiated toward ECs on the graft lumen, coexpressing CD14 and eNOS. Right underneath the luminal layer, cells expressed CD14 and αSMA but not eNOS, suggesting that MC could also differentiate into SMC. Given the position of cells expressing eNOS (lumen) versus αSMA (underneath the lumen), it is possible that differentiation of MC into vascular cells might be dependent on the type of mechanical forces acting on luminal cells (shear stress) versus the underlying layers (cyclic pressure). Indeed, our previous work showed that application of shear stress induced differentiation of MC-EC into arterial EC, as evidenced by downregulation of venous genes, upregulation of arterial genes and production of NO. More experiments are this interesting hypothesis in the future.

REFERENCES (all of which are herein entirely incorporated by reference): Mozaffarian, D., Benjamin, E. J., Go, A. S., Arnett, D. K., Blaha, M. J., Cushman, M., De Ferranti, S., Després, J.-P., Fullerton, H. J. & Howard, V. J. Heart disease and stroke statistics—2015 update: a report from the American Heart Association. circulation 131, e29-e322 (2015); Xu, S., Lu, F., Cheng, L., Li, C., Zhou, X., Wu, Y., Chen, H., Zhang, K., Wang, L. & Xia, J. Preparation and characterization of small-diameter decellularized scaffolds for vascular tissue engineering in an animal model. Biomedical engineering online 16, 1-15 (2017); Quint, C., Arief, M., Muto, A., Dardik, A. & Niklason, L. E. Allogeneic human tissue-engineered blood vessel. Journal of vascular surgery 55, 790-798 (2012); Dahl, S. L., Kypson, A. P., Lawson, J. H., Blum, J. L., Strader, J. T., Li, Y., Manson, R. J., Tente, W. E., DiBernardo, L. & Hensley, M. T. Readily available tissue-engineered vascular grafts. Science translational medicine 3, 68ra69-68ra69 (2011); Syedain, Z. H., Meier, L. A., Lahti, M. T., Johnson, S. L. & Tranquillo, R. T. Implantation of completely biological engineered grafts following decellularization into the sheep femoral artery. Tissue Engineering Part A 20, 1726-1734 (2014); Syedain, Z., Reimer, J., Lahti, M., Berry, J., Johnson, S., Bianco, R. & Tranquillo, R. T. Tissue engineering of acellular vascular grafts capable of somatic growth in young lambs. Nature communications 7, 1-10 (2016); Wu, W., Allen, R. A. & Wang, Y. Fast-degrading elastomer enables rapid remodeling of a cell-free synthetic graft into a neoartery. Nature medicine 18, 1148-1153 (2012); Filipe, E. C., Santos, M., Hung, J., Lee, B. S., Yang, N., Chan, A. H., Ng, M. K., Rnjak-Kovacina, J. & Wise, S. G. Rapid endothelialization of off-the-shelf small diameter silk vascular grafts. JACC: Basic to Translational Science 3, 38-53 (2018); Ran, X., Ye, Z., Fu, M., Wang, Q., Wu, H., Lin, S., Yin, T., Hu, T. & Wang, G. Design, preparation, and performance of a novel bilayer tissue-engineered small-diameter vascular graft. Macromolecular bioscience 19, 1800189 (2019); Kaushal, S., Amiel, G. E., Guleserian, K. J., Shapira, O. M., Perry, T., Sutherland, F. W., Rabkin, E., Moran, A. M., Schoen, F. J. & Atala, A. Functional small-diameter neovessels created using endothelial progenitor cells expanded ex vivo. Nature medicine 7, 1035-1040 (2001); De Visscher, G., Mesure, L., Meuris, B., Ivanova, A. & Flameng, W. Improved endothelialization and reduced thrombosis by coating a synthetic vascular graft with fibronectin and stem cell homing factor SDF-1α. Acta biomaterialia 8, 1330-1338 (2012); Mahara, A., Somekawa, S., Kobayashi, N., Hirano, Y., Kimura, Y., Fujisato, T. & Yamaoka, T. Tissue-engineered acellular small diameter long-bypass grafts with neointima-inducing activity. Biomaterials 58, 54-62 (2015); Duan, Y., Yu, S., Xu, P., Wang, X., Feng, X., Mao, Z. & Gao, C. Co-immobilization of CD133 antibodies, vascular endothelial growth factors, and REDV peptide promotes capture, proliferation, and differentiation of endothelial progenitor cells. Acta biomaterialia 96, 137-148 (2019); Xing, Z., Wu, S., Zhao, C., Bai, Y., Jin, D., Yin, M., Liu, H. & Fan, Y. Vascular transplantation with dual-biofunctional ePTFE vascular grafts in a porcine model. Journal of Materials Chemistry B 9, 7409-7422 (2021); Yu, J., Wang, A., Tang, Z., Henry, J., Lee, B. L.-P., Zhu, Y., Yuan, F., Huang, F. & Li, S. The effect of stromal cell-derived factor-1α/heparin coating of biodegradable vascular grafts on the recruitment of both endothelial and smooth muscle progenitor cells for accelerated regeneration. Biomaterials 33, 8062-8074 (2012); Talacua, H., Smits, A. I., Muylaert, D. E., Van Rijswijk, J. W., Vink, A., Verhaar, M. C., Driessen-Mol, A., Van Herwerden, L. A., Bouten, C. V. & Kluin, J. In situ tissue engineering of functional small-diameter blood vessels by host circulating cells only. Tissue Engineering Part A 21, 2583-2594 (2015); Tan, R. P., Chan, A. H., Wei, S., Santos, M., Lee, B. S., Filipe, E. C., Akhavan, B., Bilek, M. M., Ng; M. K. & Xiao, Y. Bioactive materials facilitating targeted local modulation of inflammation. JACC: Basic to Translational Science 4, 56-71 (2019); Smith Jr, R. J., Koobatian, M. T., Shahini, A., Swartz, D. D. & Andreadis, S. T. Capture of endothelial cells under flow using immobilized vascular endothelial growth factor. Biomaterials 51, 303-312 (2015); Koobatian, M. T., Row, S., Smith Jr, R. J., Koenigsknecht, C., Andreadis, S. T. & Swartz, D. D. Successful endothelialization and remodeling of a cell-free small-diameter arterial graft in a large animal model. Biomaterials 76, 344-358 (2016); Nasiri, B., Row, S., Smith Jr, R. J., Swartz, D. D. & Andreadis, S. T. Cell-Free Vascular Grafts That Grow with the Host. Advanced Functional Materials 30, 2005769 (2020); Smith Jr, R. J., Yi, T., Nasiri, B., Breuer, C. K. & Andreadis, S. T. Implantation of VEGF-functionalized cell-free vascular grafts: regenerative and immunological response. The FASEB Journal 33, 5089-5100 (2019); Smith, R. J., Nasiri, B., Kann, J., Yergeau, D., Bard, J. E., Swartz, D. D. & Andreadis, S. T. Endothelialization of arterial vascular grafts by circulating monocytes. Nature communications 11, 1-16 (2020); Manka, D. R., Wiegman, P., Din, S., Sanders, J. M., Green, S. A., Gimple, L. W., Ragosta, M., Powers, E. R., Ley, K. & Sarembock, I. J. Arterial Injury Increases Expression of Inflammatory Adhesion Molecules in the Carotid Arteries of Apolipoprotein-E-Deficient Micel. Journal of vascular research 36, 372-378 (1999); Etingin, O. R., Silverstein, R. L. & Hajjar, D. P. Identification of a monocyte receptor on herpesvirus-infected endothelial cells. Proceedings of the National Academy of Sciences 88, 7200-7203 (1991); Mould, A., Komoriya, A., Yamada, K. & Humphries, M. The CS5 peptide is a second site in the IIICS region of fibronectin recognized by the integrin alpha 4 beta 1. Inhibition of alpha 4 beta 1 function by RGD peptide homologues. Journal of Biological Chemistry 266, 3579-3585 (1991); Lobb, R. R. & Hemler, M. E. The pathophysiologic role of alpha 4 integrins in vivo. The Journal of clinical investigation 94, 1722-1728 (1994); Humphries, M. J., Komoriya, A., Akiyama, S., Olden, K. & Yamada, K. Identification of two distinct regions of the type Ill connecting segment of human plasma fibronectin that promote cell type-specific adhesion. Journal of Biological Chemistry 262, 6886-6892 (1987); Huo, Y., Hafezi-Moghadam, A. & Ley, K. Role of vascular cell adhesion molecule-1 and fibronectin connecting segment-1 in monocyte rolling and adhesion on early atherosclerotic lesions. Circulation research 87, 153-159 (2000); Livet, J., Weissman, T. A., Kang, H., Draft, R. W., Lu, J., Bennis, R. A., Sanes, J. R. & Lichtman, J. W. Transgenic strategies for combinatorial expression of fluorescent proteins in the nervous system. Nature 450, 56-62 (2007); Snippert, H. J., van der Flier, L. G., Sato, T., van Es, J. H., van den Born, M., Kroon-Veenboer, C., Barker, N., Klein, A. M., van Rheenen, J., Simons, B. D. & Clevers, H. Intestinal crypt homeostasis results from neutral competition between symmetrically dividing Lgr5 stem cells. Cell 143, 134-144 (2010); Jung, S., Aliberti, J., Graemmel, P., Sunshine, M. J., Kreutzberg, G. W., Sher, A. & Littman, D. R. Analysis of fractalkine receptor CX(3)CR1 function by targeted deletion and green fluorescent protein reporter gene insertion. Mol Cell Biol 20, 4106-4114 (2000); Nolfi, A. L., Behun, M. N., Yates, C. C., Brown, B. N. & Kulkarni, M. Beyond Growth Factors: Macrophage-Centric Strategies for Angiogenesis. Current Pathobiology Reports, 1-10 (2020); Jin, H., Su, J., Garmy-Susini, B., Kleeman, J. & Varner, J. Integrin α4β1 promotes monocyte trafficking and angiogenesis in tumors. Cancer research 66, 2146-2152 (2006); Roh, J. D., Sawh-Martinez, R., Brennan, M. P., Jay, S. M., Devine, L., Rao, D. A., Yi, T., Mirensky, T. L., Nalbandian, A. & Udelsman, B. Tissue-engineered vascular grafts transform into mature blood vessels via an inflammation-mediated process of vascular remodeling. Proceedings of the National Academy of Sciences 107, 4669-4674 (2010); Gupta, P., Chaudhuri, G. R., Janani, G., Agarwala, M., Ghosh, D., Nandi, S. K. & Mandal, B. B. Functionalized Silk Vascular Grafts with Decellularized Human Wharton's Jelly Improves Remodeling via Immunomodulation in Rabbit Jugular Vein. Advanced Healthcare Materials, 2100750 (2021); Hibino, N., Yi, T., Duncan, D. R., Rathore, A., Dean, E., Naito, Y., Dardik, A., Kyriakides, T., Madri, J. & Pober, J. S. A critical role for macrophages in neovessel formation and the development of stenosis in tissue-engineered vascular grafts. The FASEB Journal 25, 4253-4263 (2011); Danenberg, H. D., Fishbein, I., Gao, J., Mönkkönen, J., Reich, R., Gati, I., Moerman, E. & Golomb, G. Macrophage depletion by clodronate-containing liposomes reduces neointimal formation after balloon injury in rats and rabbits. Circulation 106, 599-605 (2002); Chopra, H., Hung, M., Kwong, D., Zhang, C. & Pow, E. Insights into endothelial progenitor cells: origin, classification, potentials, and prospects. Stem cells international 2018(2018); Wang, B., Wang, W., Yu, Y., Zhang, Y., Zhang, J. & Yuan, Z. The study of angiogenesis stimulated by multivalent peptide ligand-modified alginate. Colloids and Surfaces B: Biointerfaces 154, 383-390 (2017); Massia, S. P. & Hubbell, J. A. Vascular endothelial cell adhesion and spreading promoted by the peptide REDV of the IIICS region of plasma fibronectin is mediated by integrin alpha 4 beta 1. Journal of Biological Chemistry 267, 14019-14026 (1992); Hubbell, J. A., Massia, S. P., Desai, N. P. & Drumheller, P. D. Endothelial cell-selective materials for tissue engineering in the vascular graft via a new receptor. Bio/technology 9, 568-572 (1991); Hirsh, J. Heparin. New England Journal of Medicine 324, 1565-1574 (1991); Ji, Y., Wei, Y., Liu, X., Wang, J., Ren, K. & Ji, J. Zwitterionic polycarboxybetaine coating functionalized with REDV peptide to improve selectivity for endothelial cells. Journal of Biomedical Materials Research Part A 100, 1387-1397 (2012); Wen, C., Zhang, J., Li, Y., Zheng, W., Liu, M., Zhu, Y., Sui, X., Zhang, X., Han, Q. & Lin, Y. A zwitterionic hydrogel coated titanium surface with high-efficiency endothelial cell selectivity for rapid re-endothelialization. Biomaterials Science 8, 5441-5451 (2020); Mahara, A., Kitagawa, K., Otaka, A., Nakaoki, T., Ishihara, K. & Yamaoka, T. Impact of REDV peptide density and its linker structure on the capture, movement, and adhesion of flowing endothelial progenitor cells in microfluidic devices. Materials Science and Engineering: C, 112381 (2021); Devalliere, J., Chen, Y., Dooley, K., Yarmush, M. L. & Uygun, B. E. Improving functional re-endothelialization of acellular liver scaffold using REDV cell-binding domain. Acta biomaterialia 78, 151-164 (2018); Wang, W., Guo, L., Yu, Y., Chen, Z., Zhou, R. & Yuan, Z. Peptide REDV-modified polysaccharide hydrogel with endothelial cell selectivity for the promotion of angiogenesis. Journal of Biomedical Materials Research Part A 103, 1703-1712 (2015); Mattila, J. T., Beaino, W., Maiello, P., Coleman, M. T., White, A. G., Scanga, C. A., Flynn, J. L. & Anderson, C. J. Positron emission tomography imaging of macaques with tuberculosis identifies temporal changes in granuloma glucose metabolism and integrin α4β1-expressing immune cells. The Journal of Immunology 199, 806-815 (2017); Gerszten, R. E., Lim, Y.-C., Ding, H. T., Snapp, K., Kansas, G., Dichek, D. A., Cabanas, C., Sánchez-Madrid, F., Gimbrone Jr, M. A. & Rosenzweig, A. Adhesion of monocytes to vascular cell adhesion molecule-1-transduced human endothelial cells: Implications for atherogenesis. Circulation research 82, 871-878 (1998); Chan, J. R., Hyduk, S. J. & Cybulsky, M. I. Chemoattractants Induce a Rapid and Transient Upregulation of Monocyte α4 Integrin Affinity for Vascular Cell Adhesion Molecule 1 Which Mediates Arrest An Early Step in the Process of Emigration. Journal of Experimental Medicine 193, 1149-1158 (2001); Ogle, M. E., Segar, C. E., Sridhar, S. & Botchwey, E. A. Monocytes and macrophages in tissue repair: Implications for immunoregenerative biomaterial design. Experimental Biology and Medicine 241, 1084-1097 (2016); Shi, C. & Pamer, E. G. Monocyte recruitment during infection and inflammation. Nature reviews immunology 11, 762-774 (2011); Tahir, S. & Steffens, S. Nonclassical monocytes in cardiovascular physiology and disease. American Journal of Physiology-Cell Physiology 320, C761-C770 (2021); Getzin, T., Krishnasamy, K., Gamrekelashvili, J., Kapanadze, T., Limbourg, A., Häger, C., Napp, L. C., Bauersachs, J., Haller, H. & Limbourg, F. P. The chemokine receptor CX 3 CR 1 coordinates monocyte recruitment and endothelial regeneration after arterial injury. EMBO molecular medicine 10, 151-159 (2018); Spiller, K. L., Anfang, R. R., Spiller, K. J., Ng, J., Nakazawa, K. R., Daulton, J. W. & Vunjak-Novakovic, G. The role of macrophage phenotype in vascularization of tissue engineering scaffolds. Biomaterials 35, 4477-4488 (2014); Panek, C. A., Ramos, M. V., Mejias, M. P., Abrey-Recalde, M. J., Fernandez-Brando, R. J., Gori, M. S., Salamone, G. V. & Palermo, M. S. Differential expression of the fractalkine chemokine receptor (CX3CR1) in human monocytes during differentiation. Cellular & molecular immunology 12, 669-680 (2015); Iqbal, A. J., McNeill, E., Kapellos, T. S., Regan-Komito, D., Norman, S., Burd, S., Smart, N., Machemer, D. E., Stylianou, E. & McShane, H. Human CD68 promoter GFP transgenic mice allow analysis of monocyte to macrophage differentiation in vivo. Blood, The Journal of the American Society of Hematology 124, e33-e44 (2014); and Soehnlein, O. Macrophages shine bright. Blood, The Journal of the American Society of Hematology 124, 2320-2322 (2014).

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

1. A synthetic peptide comprising an amino acid sequence having at least 90 percent sequence identity to SEQ ID NO: 2.

2. (canceled)

3. An implantable vascular graft material, comprising: a substrate comprising a graft material, the substrate defining a top surface and a bottom surface; and one or more bispecific binding partners having a luminal binding domain bound to the top surface and one or more cellular binding domains.

4. The implantable vascular graft material of claim 3, wherein the one or more cellular binding domains are selected from monocyte binding domains, endothelial-like binding domains, and combinations thereof.

5. The implantable vascular graft material of claim 3, wherein one or more subluminal binding domains are disposed upon the bottom surface.

6. The implantable vascular graft material of claim 3, wherein the one or more cellular binding domains are monocyte binding domains including integrin α4β1 and/or VEGFR1.

7. The implantable vascular graft material of claim 3, wherein the one or more cellular binding domains are endothelial-like binding domains including Flk1.

8. The implantable vascular graft material of claim 3, wherein the luminal binding domain is characterized as an ECM binding domain, a heparin binding domain, or a collagen binding domain.

9. The implantable vascular graft material of claim 3, wherein the bispecific binding partner is a polymer, or synthetic polymer.

10. The implantable vascular graft material of claim 3, wherein the bispecific binding partner is a biopolymer or synthetic biopolymer.

11. The implantable vascular graft material of claim 3, wherein the bispecific binding partner is an aptamer or synthetic aptamer, or combination of synthetic aptamers.

12. The implantable vascular graft material of claim 3, wherein the bispecific binding partner is a peptide having at least 80 percent sequence identity to SEQ ID. No. 2.

13. The implantable vascular graft material of claim 3, wherein the bispecific binding partner is a peptide having at least 90%, at least 95%, at least 97%, or at least 99 percent sequence identity to SEQ ID. No. 2.

14. The implantable vascular graft material of claim 3, wherein the one or more one or more cellular binding domains are monocyte binding domains configured to recruit monocytes when contacted with blood.

15. (canceled)

16. An implantable vascular graft comprising: a tubular base layer comprising a graft material, the tubular base layer defining a luminal surface and an abluminal surface; and one or more bispecific binding partners having a luminal binding domain bound to the luminal surface and one or more monocyte binding domains.

17. The implantable vascular graft of claim 16, wherein the bispecific binding partner is one or more of a polymer, a biopolymer, an aptamer, or combinations thereof.

18. The implantable vascular graft of claim 16, wherein the bispecific binding partner is a fusion or multi-domain peptide having a luminal binding domain characterized as a heparin binding domain.

19. The implantable vascular graft of claim 16, wherein a chitosan-heparin layer is disposed directly atop the luminal surface and between the luminal surface and the fusion or multi-domain peptide.

20. The implantable vascular graft of claim 16, wherein the one or more bispecific binding partners is a fusion protein is characterized by the formula H2R5, wherein H2 is a heparin binding domain of fibronectin, and R5 includes a plurality of tandem repeats, wherein each tandem repeat comprises a flexible linker motif followed by a peptide comprising the sequence HIPREDVDYH.

21-43. (canceled)

44. The implantable vascular graft of claim 16, wherein the bispecific binding partner is a peptide having at least 90 percent sequence identity to SEQ ID. No. 2.

45. The implantable vascular graft of claim 16, wherein the bispecific binding partner is a peptide having at least 90%, at least 95%, at least 97%, or at least 99 percent sequence identity to SEQ ID. No. 2.
